(19) United States
(12) Patent Application Publication
(10) Pub. No.: US 2025/0235310 A1
(43) Pub. Date: Jul. 24, 2025

(54) PROSTHETIC VALVE DELIVERY APPARATUS

(71) Applicant: EDWARDS LIFESCIENCES CORPORATION, Irvine, CA (US)

(72) Inventors: Grant Jason Gangeness, Carlsbad, CA (US); Izaak Rosen, Irvine, CA (US); Elazar Levi Schwarcz, Netanya (IL); Natanel Simcha Sirote, Zikhron Ya’akov (IL)

(73) Assignee: EDWARDS LIFESCIENCES CORPORATION, Irvine, CA (US)

(21) Appl. No.: 19/170,412

(22) Filed: Apr. 4, 2025

Related U.S. Application Data
(63) Continuation of application No. PCT/US2023/035374, filed on Oct. 18, 2023.
(60) Provisional application No. 63/459,820, filed on Apr. 17, 2023.
(60) Provisional application No. 63/417,971, filed on Oct. 20, 2022.

(51) Int. Cl. A61F 2/24

(52) U.S. Cl. CPC A61F 2/2433

(57) ABSTRACT

A delivery apparatus configured to deliver a prosthetic valve can include a handle, a first shaft extending distally from the handle, a second shaft extending through a lumen of the first shaft, a nose cone mounted on a distal end portion of the second shaft, a balloon mounted along the distal end portion of the second shaft, and a first pull wire extending longitudinally along the second shaft. A distal end of the first pull wire is fixed relative to the second shaft at a location that is distal to a distal end of the balloon. Tension in the first pull wire can be adjusted to adjust a curvature of the distal end portion of the second shaft and the nose cone.

### CROSS-REFERENCE TO RELATED APPLICATIONS

This application is a continuation of International Patent Application No. PCT/US2023/035374, filed Oct. 18, 2023, which claims the benefit of U.S. Provisional Application No. 63/417,971, filed Oct. 20, 2022, and U.S. Provisional Application No. 63/459,820, filed Apr. 17, 2023, all of which applications are incorporated by reference herein in their entireties.

### FIELD

The present disclosure concerns delivery apparatuses, systems, and methods for implantation of a prosthetic valve.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (for example, stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient's vasculature (for example, through a femoral artery and the aorta) until the prosthetic heart valve reaches the implantation site in the heart. The prosthetic heart valve is then expanded to its functional size, for example, by inflating a balloon on which the prosthetic valve is mounted, actuating a mechanical actuator that applies an expansion force to the prosthetic heart valve, or by deploying the prosthetic heart valve from a sheath of the delivery apparatus so that the prosthetic heart valve can self-expand to its functional size. Operating a delivery apparatus for implantation of a prosthetic heart valve involves complex steps and requires specialized skills. Accordingly, improvements to known transcatheter delivery apparatuses to facilitate their operation are desirable.

### SUMMARY

Described herein are systems and methods for delivering prosthetic devices, such as prosthetic heart valves, through the body and into the heart for implantation therein. The prosthetic devices delivered with the delivery systems disclosed herein are, for example, radially expandable from a radially compressed state mounted on the delivery system to a radially expanded state for implantation using an inflatable balloon (or equivalent expansion device) of the delivery system. Exemplary delivery routes through the body and into the heart include transfemoral routes, transapical routes, and transaortic routes, among others. Although the devices and methods disclosed herein are particular suited for implanting prosthetic heart valves (for example, a prosthetic aortic valve or prosthetic mitral valve), the disclosed devices and methods can be adapted for implanting other types of prosthetic valves within the body (for example, prosthetic venous valves) or other types of expandable prosthetic devices adapted to be implanted in various body lumens.

In one aspect, a delivery apparatus for a prosthetic implant can comprise a handle and one or more shafts coupled to the handle. In addition to these components, a delivery apparatus can further comprise one or more of the components disclosed herein.

In some examples, a delivery apparatus can comprise a first shaft extending distally from the handle and a second shafting extending through a lumen of the first shaft.

In some examples, the delivery apparatus can further include a nose cone mounted on a distal end portion of the second shaft, a balloon mounted along the distal end portion of the second shaft, and a first pull wire extending longitudinally along the second shaft.

In some examples, a distal end of the first pull wire is fixed relative to the second shaft at a location that is distal to a distal end of the balloon. Tension in the first pull wire can be adjusted to adjust a curvature of the distal end portion of the second shaft and the nose cone.

In some examples, a delivery apparatus can comprise a handle, a first shaft extending distally from the handle, a second shaft extending through a lumen of the first shaft, a third shaft extending through a lumen of the second shaft, a nose cone mounted on a distal end portion of the third shaft, a first pull wire extending longitudinally along the third shaft, and a second pull wire extending longitudinally along the first shaft.

In some examples, a distal end of the first pull wire is fixedly connected to the nose cone and a distal end of the second pull wire is fixedly connected to a distal end portion of the first shaft. Tensioning the first pull wire can be configured to adjust a curvature of the nose cone. Tensioning the second pull wire can be configured to adjust a curvature of the distal end portion of the first shaft. The distal end of the first pull wire is distal to the distal end of the second pull wire.

In certain aspects, a method can comprise receiving a delivery apparatus that includes a handle, a first shaft extending distally from the handle, a second shaft extending through a lumen of the first shaft, a third shaft extending through a lumen of the second shaft, a nose cone mounted on a distal end portion of the third shaft, a first pull wire extending longitudinally along the third shaft, and a second pull wire extending longitudinally along the first shaft. The method can further comprise increasing tension in the second pull wire to adjust a curvature of the distal end portion of the first shaft, and increasing tension in the first pull wire to adjust a curvature of the nose cone.

In certain aspects, a method of implanting a prosthetic valve can comprise introducing a delivery apparatus into a vasculature of a subject. The prosthetic valve in a radially compressed configuration can be retained on a distal end portion of the delivery apparatus. The method can further comprise adjusting a curvature of the distal end portion of the shaft to navigate the prosthetic valve through an arched region of the vasculature, positioning the prosthetic valve within or adjacent an annulus of a native heart valve, retracting the shaft relative to the prosthetic valve so as to expose the prosthetic valve, adjusting a curvature of a nose cone of the delivery apparatus until the prosthetic valve is substantially coaxial with the annulus of the native heart valve, and radially expanding the prosthetic valve.

In some examples, adjusting the curvature of the distal end portion of the shaft can comprise tensioning a first pull wire. A distal end of the first pull wire can be fixedly connected to the distal end portion of the shaft. In some examples, adjusting the curvature of the nose cone can comprise tensioning a second pull wire. A distal end of the second pull wire can be fixedly connected to the nose cone. The distal end of the first pull wire can be proximal to the prosthetic valve, and the distal end of the second pull wire can be distal to the prosthetic valve.

The above method(s) can be performed on a living animal or on a simulation, such as on a cadaver, cadaver heart, anthropomorphic ghost, simulator (for example, with body parts, heart, tissue, etc. being simulated).

In some examples, a delivery apparatus can comprise a handle, a shaft extending distally from the handle, a nose cone mounted on a distal end portion of the shaft, and a pull wire extending longitudinally along the shaft. A distal end of the pull wire can be fixedly connected to a distal end of the nose cone. Tension in the first pull wire can be adjusted to adjust a curvature of the distal end portion of the shaft and the nose cone.

In some examples, a delivery apparatus comprises one or more of the components recited in Examples 1-17 and 24-30 described in the section “Additional Examples of the Disclosed Technology” below.

The foregoing and other objects, features, and advantages of the disclosed technology will become more apparent from the following detailed description, which proceeds with reference to the accompanying figures.

### BRIEF DESCRIPTION OF THE DRAWINGS

FIG. 1 is a side view of a delivery apparatus for implanting a prosthetic heart valve, according to one example.

FIG. 2A is a cross-sectional view of the handle of the delivery apparatus of FIG. 1.

FIG. 2B is another cross-sectional view of the handle of the delivery apparatus of FIG. 1.

FIG. 3 is side view of a section of the handle and a section of the distal end portion of the delivery apparatus of FIG. 1.

FIG. 4 is a side view of the distal end portion of the delivery apparatus of FIG. 1.

FIG. 5 is a side cross-sectional view of the distal end portion of the delivery apparatus of FIG. 1 showing the balloon in an inflated state.

FIG. 6 is an enlarged perspective view of a collet used in the handle of the delivery apparatus of FIG. 1.

FIG. 7 is a cross-sectional view of the collet of FIG. 6.

FIG. 8 is an enlarged side view of a mounting member for a prosthetic heart valve.

FIGS. 9-11 are enlarged, cross-sectional views of the distal end portion of the delivery apparatus of FIG. 1, showing the inflation of a balloon for deployment of a prosthetic heart valve on the balloon.

FIG. 12 depicts alignment of a prosthetic valve mounted on a balloon with an aortic annulus, according to one example.

FIG. 13 depicts a guide wire shaft for a delivery apparatus, wherein a nose cone is mounted on the guide wire shaft and a pull wire extends along the guide wire shaft, according to one example.

FIG. 13A depicts a cross-sectional view of the guide wire shaft of FIG. 13 taken along line 13A-13A.

FIG. 14 depicts a Y-connector for a delivery apparatus housing a sliding member connected to the pull wire of FIG. 13, according to one example.

FIG. 14A depicts a cross-sectional view of the Y-connector of FIG. 14 taken along line 14A-14A.

FIG. 14B depicts the sliding member of the Y-connector of FIG. 13.

FIG. 15 depicts a Y-connector for a delivery apparatus, according to another example.

FIG. 15A depicts a cross-sectional view of the Y-connector of FIG. 15 taken along line 15A-15A.

FIG. 16A depicts a longitudinal cross-section view of a nose cone, according to another example.

FIG. 16B illustrates bending of a distal portion of the nose cone of FIG. 16A.

FIG. 16C depicts a radial cross-section view of the nose cone of FIG. 16A, taken along the line 16C-16C.

FIG. 16D depicts a radial cross-section view of the nose cone of FIG. 16A, taken along the line 16D-16D.

FIG. 17A depicts a longitudinal cross-section view of a nose cone, according to another example.

FIG. 17B depicts a longitudinal cross-section view of a nose cone, according to another example.

FIG. 17C depicts a radial cross-section view of the nose cone of FIG. 17A, taken along the line 17C-17C.

FIG. 17D depicts a radial cross-section view of the nose cone of FIG. 17A, taken along the line 17D-17D.

FIG. 17E depicts a radial cross-section view of the nose cone of FIG. 17B, taken along the line 17E-17E.

FIG. 18 depicts an example of a prosthetic heart valve that can be implanted using any of the delivery apparatuses disclosed herein.

### DETAILED DESCRIPTION

### General Considerations

It should be understood that the disclosed examples can be adapted to deliver and implant prosthetic devices in any of the native annuluses of the heart (for example, the pulmonary, mitral, and tricuspid annuluses), and can be used with any of various delivery approaches (for example, retrograde, antegrade, transseptal, transventricular, transatrial, etc.).

For purposes of this description, certain aspects, advantages, and novel features of the examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved. The technologies from any example can be combined with the technologies described in any one or more of the other examples. In view of the many possible examples to which the principles of the disclosed technology may be applied, it should be recognized that the illustrated examples are only preferred examples and should not be taken as limiting the scope of the disclosed technology.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the terms “coupled” and “connected” generally mean electrically, electromagnetically, and/or physically (for example, mechanically or chemically) coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (for example, out of the patient's body), while distal motion of the device is motion of the device away from the user and toward the implantation site (for example, into the patient's body). The terms “longitudinal” and “axial” refer to an axis extending in the proximal and distal directions, unless otherwise expressly defined.

As described herein, the term “inflow” can generally refer to a position, direction, or portion of the prosthetic heart valve that is closer to an inlet into which blood flow enters the prosthetic heart valve. As described herein, the term “outflow” can generally refer to a position, direction, or portion of a prosthetic heart valve that is closer to an outlet from which blood flow exits the prosthetic heart valve.

Directions and other relative references (for example, inner, outer, upper, lower, etc.) may be used to facilitate discussion of the drawings and principles herein, but are not intended to be limiting. For example, certain terms may be used such as “inside,” “outside,”, “top,” “down,” “interior,” “exterior,” and the like. Such terms are used, where applicable, to provide some clarity of description when dealing with relative relationships, particularly with respect to the illustrated examples. Such terms are not, however, intended to imply absolute relationships, positions, and/or orientations. For example, with respect to an object, an “upper” part can become a “lower” part simply by turning the object over. Nevertheless, it is still the same part and the object remains the same. As used herein, “and/or” means “and” or “or,” as well as “and” and “or.”

### Overview of an Exemplary Delivery Apparatus for Implanting a Prosthetic Valve

A delivery apparatus for implanting a prosthetic, transcatheter heart valve via a patient's vasculature can include an adjustment device for adjusting the position of a balloon relative to a crimped prosthetic valve (and/or vice versa). A balloon catheter can extend coaxially with a guide (or flex) catheter, and a balloon at a distal end of the balloon catheter can be positioned proximal or distal to a crimped prosthetic valve. As described below in more detail, the balloon and the crimped prosthetic valve can enter the vasculature of a patient through an introducer sheath and, once the balloon and the crimped prosthetic valve reach a suitable location in the body, the relative position of the prosthetic valve and balloon can be adjusted so that the balloon is positioned within a frame of the prosthetic valve so that the prosthetic valve eventually can be expanded at the treatment site. Once the crimped prosthetic valve is positioned on the balloon, the prosthetic valve is advanced to the vicinity of the deployment location (for example, the native aortic valve) and the adjustment device can further be used to accurately adjust or “fine tune” the position of the prosthetic valve relative to the desired deployment location.

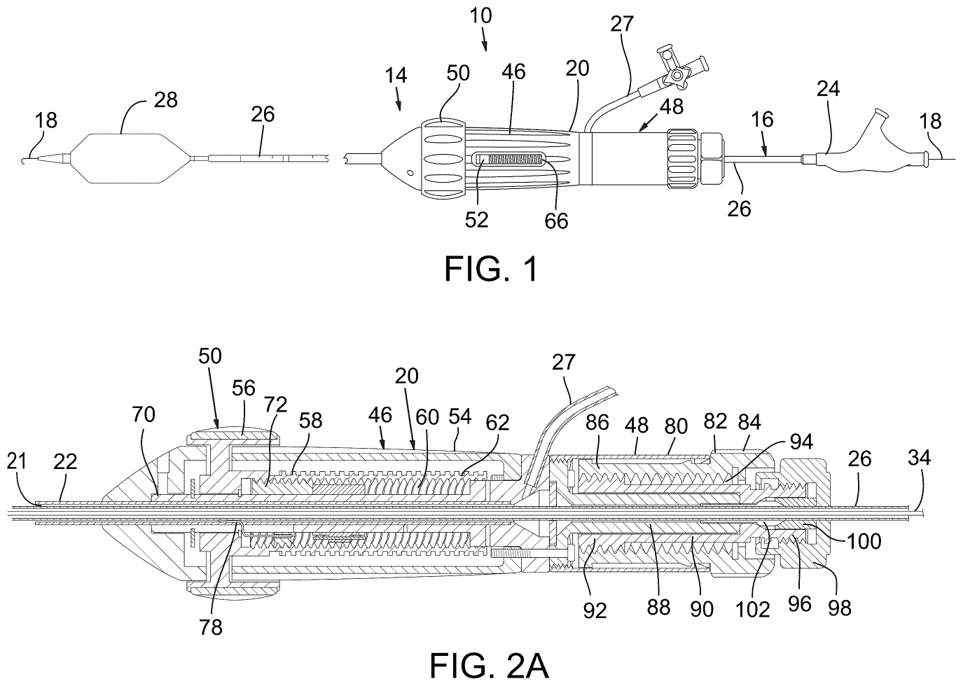

FIG. 1 shows a delivery apparatus 10 adapted to deliver a prosthetic heart valve 12 (shown schematically in FIGS. 9-11) (for example, a prosthetic aortic valve) to a heart, according to one example. The apparatus 10 generally includes a steerable guide catheter 14 (FIG. 3), and a balloon catheter 16 extending through the guide catheter 14. The guide catheter can also be referred to as a flex catheter or a main catheter. The use of the term main catheter should be understood, however, to include flex or guide catheters, as well as other catheters that do not have the ability to flex or guide through a patient's vasculature.

The guide catheter 14 and the balloon catheter 16 in the illustrated example are adapted to slide longitudinally relative to each other to facilitate delivery and positioning of prosthetic valve 12 at an implantation site in a patient's body, as described in detail below.

The guide catheter 14 includes a handle portion 20 (or simply “handle”) and an elongated guide tube, or guide catheter shaft, 22 extending distally from the handle portion 20 (FIG. 3). FIG. 1 shows the delivery apparatus without the guide catheter shaft 22 for purposes of illustration. FIG. 3 shows the guide catheter shaft 22 extending from the handle portion 20 over the balloon catheter 16. The balloon catheter 16 includes a proximal portion 24 (FIG. 1) adjacent to the handle portion 20 and an elongated shaft 26 (also referred to as “balloon catheter shaft”) that extends from the proximal portion 24 and through the handle portion 20 and a lumen 21 of the guide catheter shaft 22. The handle portion 20 can include a side arm 27 having an internal passage which fluidly communicates with a lumen defined by the handle portion 20.

As shown in FIG. 4, an inflatable balloon 28 can be mounted at a distal end of the balloon catheter 16. The delivery apparatus 10 can be configured to mount the prosthetic valve 12 in a crimped state that is initially offset from (for example, proximal to or distal to) the balloon 28 for insertion of the delivery apparatus and prosthetic valve into a patient's vasculature, as described in detail in U.S. Publication No. 2009/0281619 (U.S. application Ser. No. 12/247,846, filed Oct. 8, 2008), which is incorporated herein by reference. Because the prosthetic valve 12 is crimped at a location different from the location of the balloon 28 (for example, the prosthetic valve 12 is crimped proximal to the balloon 28 in the example depicted in FIG. 4), the prosthetic valve 12 can be crimped to a lower profile than would be possible if the prosthetic valve 12 was crimped on top of balloon 28. This lower profile permits a surgeon to navigate the delivery apparatus (including the crimped valve 12) more easily through a patient's vasculature to the treatment location. The lower profile of the crimped prosthetic valve is particularly helpful when navigating through portions of the patient's vasculature which are particularly narrow, such as the iliac artery. The lower profile also allows for treatment of a wider population of patients, with enhanced safety.

As shown in FIG. 4, a nose cone 32 can be mounted at a distal end of the delivery apparatus 10 to facilitate advancement of the delivery apparatus 10 through the patient's vasculature to the implantation site. In some instances, it may be useful to have the nose cone 32 connected to a separate elongated shaft so that the nose cone 32 can move independently of other elements of delivery apparatus 10. The nose cone 32 can be formed of a variety of materials, including various plastic materials. Additional details of the nose cone 32 and its relationship with other components of the delivery apparatus 10 are described further below.

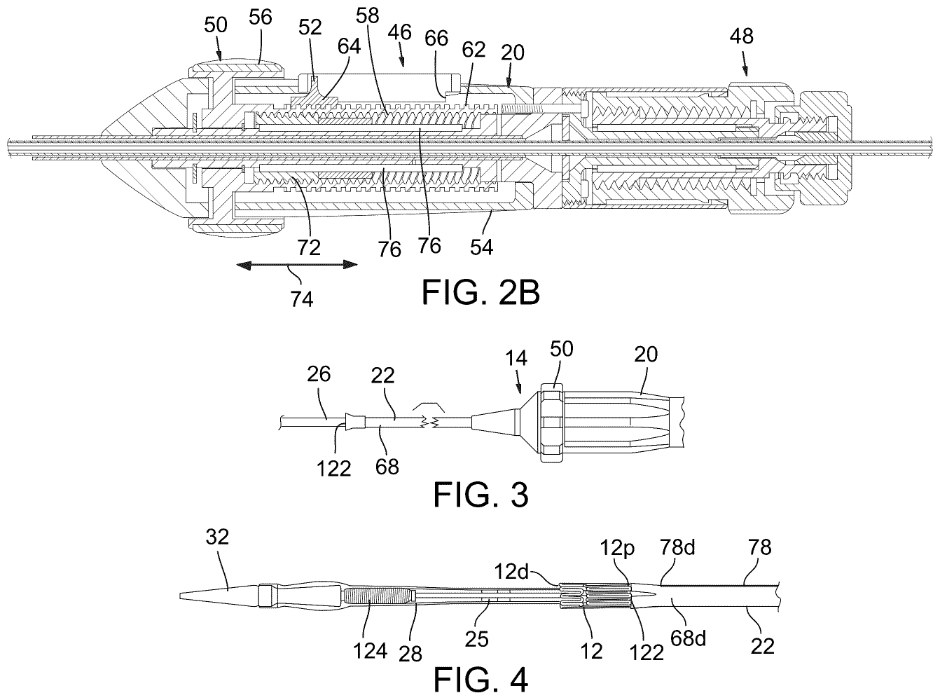

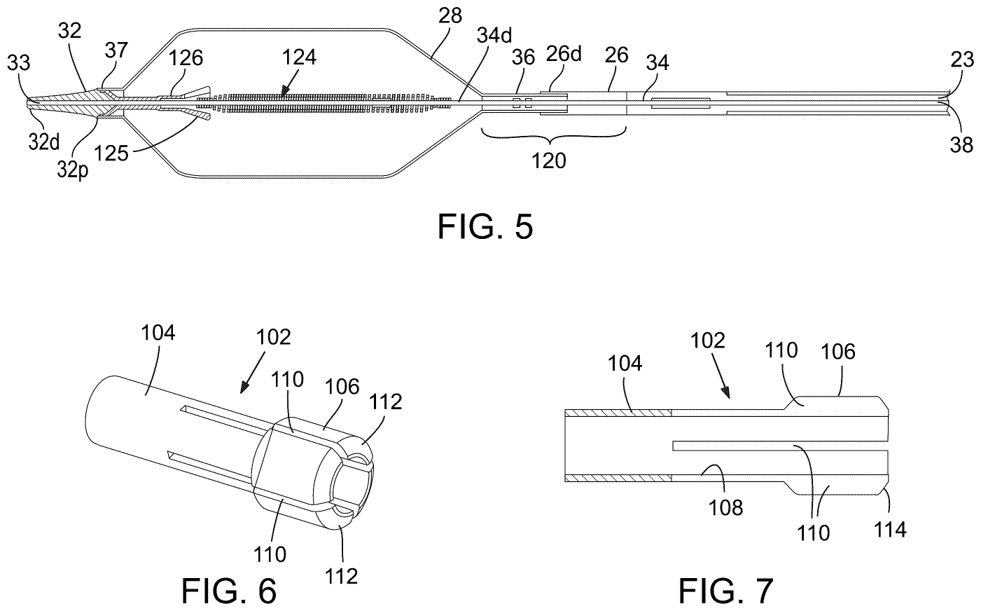

As can be seen in FIG. 5, the delivery apparatus 10 in the illustrated configuration further includes an inner shaft 34 (see also, FIG. 2A) that extends from a proximal portion 24 (also referred to as an “adaptor” or a “Y-connector”) and coaxially through a lumen 23 of the balloon catheter shaft 26 and the balloon 28. The balloon 28 can be mounted along a distal end portion 34d of the inner shaft 34. In one specific example, a proximal end 36 of the balloon 28 can be secured to a distal end 26d of balloon catheter shaft 26 (for example, with a suitable adhesive) (FIG. 5), and a distal end 37 of the balloon 28 can be secured to the nose cone 32 (for example, with a suitable adhesive) (FIG. 5). The outer diameter of inner shaft 34 can be sized such that an annular space is defined between the inner shaft 34 and the balloon catheter shaft 26 along an entire length of the balloon catheter shaft 26. The proximal portion 24 of the balloon catheter 16 can be formed with a fluid passageway (not shown) that is fluidly connectable to a fluid source (for example, a syringe containing an inflation fluid such as saline) for inflating the balloon 28. The fluid passageway is in fluid communication with the annular space between the inner shaft 34 and balloon catheter shaft 26 such that fluid from the fluid source can flow through the fluid passageway, through the annular space between the shafts 34 and 26, and into the balloon 28 to inflate the same and deploy the prosthetic valve 12.

The proximal portion 24 also defines an inner lumen that is in communication with a lumen 38 of the inner shaft 34 (FIG. 5). The lumen 38 is sized to receive a guide wire 18 (FIG. 1) that can extend coaxially through the inner shaft 34 and the nose cone 32. For example, the guide wire 18 can extend through the lumen 38 of the inner shaft 34 and a central lumen 33 of the nose cone 32. Thus, the inner shaft 34 can also be referred to as a “guide wire shaft,” and the lumen 38 can also be referred to as a “guide wire lumen.”

The guide wire shaft 34 and balloon catheter shaft 26 can be formed from any of various suitable materials, such as nylon, braided stainless steel wires, or a polyether block amide (commercially available as Pebax®). The shafts 26, 34 can have longitudinal sections formed from different materials in order to vary the flexibility of the shafts along their lengths. The guide wire shaft 34 can have an inner liner or layer formed of Teflon® to minimize sliding friction with the guide wire 18.

In certain examples, the guide wire shaft 34 can comprise an inner tubular member and a braided layer surrounding the tubular member. Similarly, the balloon catheter shaft 26 can also have an inner tubular member and an outer braided layer surrounding the tubular member. In certain examples, the braided layers can be constructed from braided metal wires (for example, stainless steel wires, Nitinol wires, etc.). In certain examples, the braided layers can be formed from metal coils (for example, a stainless-steel coil, etc.). The braided layers can have various braid density along the shafts so as to impart desired flexibility and stiffness to different parts of the shafts. In certain examples, a metal braided layer can be replaced with a stainless steel hypotube that is formed with laser-cut and circumferentially extending openings. Additional examples of the braided layer are described in U.S. Pat. No. 8,568,472, which is incorporated herein by reference in its entirety.

The guide catheter shaft 22 comprises a steerable distal end portion 68 (also referred to as the “steerable section”) (FIG. 3), the curvature of which can be adjusted by an operator to assist in guiding the delivery apparatus 10 through the patient's vasculature, and in particular, the aortic arch. The handle 20 in the illustrated embodiment comprises a distal handle portion 46 and a proximal handle portion 48. The distal handle portion 46 is configured to function as an actuation mechanism for adjusting the curvature of the steerable section 68 of the guide catheter shaft 22 and as a flex indicating device that allows the operator to measure the relative amount of flex of the steerable section 68 of the guide catheter shaft 22. In addition, the flex indicating device can provide a visual and tactile response at the handle 20, which provides the operator with an immediate and direct way to determine the amount of flex of the steerable section 68 of the guide catheter shaft 22.

The distal handle portion 46 can be operatively connected to the steerable section 68 and functions as an adjustment mechanism to permit the operator to adjust the curvature of the steerable section 68 via manual adjustment of the distal handle portion 46. Explaining further, the distal handle portion 46 comprises a flex activating member 50, an indicator pin 52, and a cylindrical main body, or housing 54. As shown in FIGS. 2A and 2B, the flex activating member 50 comprises an adjustment knob 56 and a shaft 58 extending proximally from the knob into the housing 54. A proximal end portion of the guide catheter shaft 22 extends into and is fixed within a central lumen of the housing 54. An inner sleeve 70 surrounds a portion of the guide catheter shaft 22 inside the housing 54. A threaded slide nut 72 is disposed on and is slidable relative to the inner sleeve 70. The slide nut 72 is formed with external threads that mate with internal threads 60 of the shaft 58.

The slide nut 72 can have two slots formed on the inner surface of the nut and extending the length thereof. The sleeve 70 can be formed with longitudinally extending slots that are aligned with the slots of the slide nut 72 when the slide nut is placed on the sleeve 70. Disposed in each slot is a respective elongated nut guide 76, which can be in the form of an elongated rod or pin 76. The nut guides 76 extend radially into respective slots in the slide nut 72 to prevent rotation of the slide nut 72 relative to the sleeve 70. By virtue of this arrangement, rotation of the adjustment knob 56 (either clockwise or counterclockwise) causes the slide nut 72 to move longitudinally relative to the sleeve 70 in the directions indicated by the double-headed arrow 74.

One or more pull wires 78 (FIG. 2A) couple the adjustment knob 56 to the steerable section 68 to adjust the curvature of the steerable section 68 upon rotation of the adjustment knob 56. For example, the proximal end portion of the pull wire 78 can extend into and can be secured to a retaining pin, such as by crimping the pin around the proximal end of the pull wire, which pin is disposed in a slot in the slide nut 72. The pull wire 78 extends from the pin, through the slot in the slide nut 72, a slot in the sleeve 70, and into and through a pull wire lumen in the guide catheter shaft 22. A distal end 78d of the pull wire 78 can be secured to a distal end 68d of the steerable section 68.

The pin, which retains a proximal end of the pull wire 78, is captured in the slot in the slide nut 72. Hence, when the adjustment knob 56 is rotated to move the slide nut 72 in the proximal direction, the pull wire 78 also is moved in the proximal direction. The pull wire 78 pulls the distal end 68d of the steerable section 68 back toward the distal handle portion 46, thereby bending the steerable section 68 and reducing its radius of curvature. The friction between the adjustment knob 56 and the slide nut 72 is configured to be sufficient to hold the pull wire 78 taut, thus preserving the shape of the bend in the steerable section 68 if the operator releases the adjustment knob 56. Thus, the friction between the adjustment knob 56 and the slide nut 72 can function as a locking mechanism for the pull wire 78. When the adjustment knob 56 is rotated in the opposite direction to move the slide nut 72 in the distal direction, tension in the pull wire 78 is released. The resiliency of the steerable section 68 causes the steerable section 68 to return its normal, non-deflected shape as tension on the pull wire 78 is decreased. Because the pull wire 78 is not fixedly secured to the slide nut 72 (for example, the pin can move within the slot in the nut), movement of the slide nut 72 in the distal direction does not push on the end of the pull wire 78, causing it to buckle. Instead, the pin is allowed to float within the slot of the slide nut 72 when the knob 56 is adjusted to reduce tension in the pull wire 78, preventing buckling of the pull wire 78.

In some examples, the steerable section 68 in its non-deflected shape is slightly curved and in its fully curved position, the steerable section 68 generally conforms to the shape of the aortic arch. In some examples, the steerable section 68 can be substantially straight in its non-deflected position.

The distal handle portion 46 can have other configurations that are adapted to adjust the curvature of the steerable section 68. One such alternative handle configuration is shown in co-pending U.S. Pat. No. 7,780,723, which is incorporated herein by reference in its entirety. Additional details relating to the steerable section and handle configuration discussed above can be found in U.S. Pat. No. 8,568,472, which is incorporated herein by reference in its entirety.

The shaft 58 also includes an externally threaded surface portion 62. As shown in FIG. 2B, a base portion 64 of the indicator pin 52 mates with the externally threaded surface portion 62 of the shaft 58. The shaft 58 extends into the main body 54 and the indicator pin 52 is trapped between the externally threaded surface portion 62 and the main body 54, with a portion of the indicator pin 52 extending into a longitudinal slot 66 of the handle. As the knob 56 is rotated to increase the curvature of the steerable section 68, the indicator pin 52 tracks the external threaded portion 62 of the flex activating member and moves in the proximal direction inside of the slot 66. The greater the amount of rotation of the knob 56, the further indicator pin 52 moves towards the proximal end of the proximal handle portion 46. Conversely, rotating the knob 56 in the opposite direction decreases the curvature of the steerable section 68 (that is, straightens the guide catheter shaft 22) and causes corresponding movement of the indicator pin 52 toward the distal end of the distal handle portion 46.

The outer surface of the main body 54 of the distal handle portion 46 can include visual indicia adjacent the slot 66 that indicate the amount of flex of the steerable section 68, based on the position of the indicator pin 52 relative to the visual indicia. Such indicia can identify the amount of flex in any of a variety of manners. For example, the outer surface of the main body 54 can include a series of numbers (for example, 0 to 10) adjacent the slot that indicate the amount of curvature of the steerable section 68 based on the position of the indicator pin 52 relative to the number scale.

As described above, when the delivery apparatus 10 is introduced into the vasculature of the patient, a crimped prosthetic valve 12 can be positioned proximal to the balloon 28 (FIG. 4). Prior to expansion of the balloon 28 and deployment of prosthetic valve 12 at the treatment site, the prosthetic valve 12 can be moved relative to the balloon (or vice versa) to position the crimped prosthetic valve 12 on the balloon 28 for deploying (expanding) the prosthetic valve 12. As discussed below, the proximal handle portion 48 serves as an adjustment device that can be used to move the balloon 28 proximally into position within the frame of prosthetic valve 12, and further to accurately position the balloon 28 and the prosthetic valve 12 at the desired deployment location.

As shown in FIGS. 2A and 2B, the proximal handle portion 48 comprises an outer housing 80 and an adjustment mechanism 82. The adjustment mechanism 82, which is configured to adjust the axial position of the balloon catheter shaft 26 relative to the guide catheter shaft 22, comprises an adjustment knob 84 and a shaft 86 extending distally into the housing 80. Mounted within the housing 80 on the balloon catheter shaft 26 is an inner support 88, which in turn mounts an inner shaft 90 (also referred to as a slider or sliding mechanism). The inner shaft 90 has a distal end portion 92 formed with external threads that mate with internal threads 94 that extend along the inner surface of the adjustment mechanism 82. The inner shaft 90 further includes a proximal end portion 96 that mounts a securement mechanism 98, which is configured to retain the position of the balloon catheter shaft 26 relative to the proximal handle portion 48 for use of the adjustment mechanism 82, as further described below. The inner shaft 90 can be coupled to the inner support 88 such that rotation of the shaft 86 causes the inner shaft 90 to move axially within the handle. For example, the inner support 88 can have an axially extending rod or rail that extends into slot formed in the inner surface of the inner shaft 90. The rod or rail prevents rotation of the inner shaft 90 but allows it to move axially upon rotation of the shaft 86.

The securement mechanism 98 includes internal threads that mate with external threads of the proximal end portion 96 of the inner shaft. Mounted within the proximal end portion 96 on the balloon catheter shaft 26 is a pusher element 100 and a shaft engagement member in the form of a collet 102. The collet 102 is configured to be manipulated by the securement mechanism 98 between a first state in which the collet 102 allows the balloon catheter shaft 26 to be moved freely in the longitudinal and rotational directions and a second state in which the collet 102 frictionally engages the balloon catheter shaft 26 and prevents rotational and longitudinal movement of the balloon catheter shaft 26 relative to the inner shaft 90, as further described below.

As best shown in FIGS. 6 and 7, the collet 102 comprises a distal end portion 104, an enlarged proximal end portion 106, and a lumen 108 that receives the balloon catheter shaft 26. A plurality of axially extending, circumferentially spaced slots 110 extend from the proximal end of the collet 102 to a location on the distal end portion 104, thereby forming a plurality of flexible fingers 112. The proximal end portion can be formed with a tapered end surface 114 that engages a corresponding tapered end surface of the pusher element 100 (FIG. 2A).

As noted above, the securement mechanism 98 is operable to restrain movement of the balloon catheter shaft 26 (in the axial and rotational directions) relative to the proximal handle portion 48. Explaining further, the securement mechanism 98 is movable between a proximal position (shown in FIGS. 2A and 2B) and a distal position closer to the adjacent end of the knob 84. In the proximal position, the collet 102 applies little, if any, force against the balloon catheter shaft 26, which can slide freely relative to the collet 102, the entire handle 20, and the guide catheter shaft 22. When the securement mechanism 98 is rotated so as to move to its distal position closer to knob 84, the securement mechanism 98 urges the pusher element 100 against the proximal end of the collet 102. The tapered surface of the pusher element 100 pushes against the corresponding tapered surface 114 of the collet 102, forcing the fingers 112 radially inward against the outer surface of the balloon catheter shaft 26. The holding force of the collet 102 against the balloon catheter shaft 26 locks the balloon catheter shaft 26 relative to the inner shaft 90. In the locked position, rotation of the adjustment knob 84 causes the inner shaft 90 and the balloon catheter shaft 26 to move axially relative to the guide catheter shaft 22 (either in the proximal or distal direction, depending on the direction the knob 84 is rotated).

The adjustment knob 84 can be utilized to position the prosthetic valve 12 on the balloon 28 and/or once the prosthetic valve 12 is on the balloon, to position the prosthetic valve 12 and the balloon 28 at the desired deployment site within the native valve annulus. One specific method for implanting the prosthetic valve 12 in the native aortic valve is as follows. The prosthetic valve 12 initially can be crimped on a valve retaining region 120 (FIGS. 4 and 5) of the balloon catheter shaft 26 immediately adjacent the proximal end 36 of the balloon 28 or slightly overlapping the proximal end 36 of the balloon 28. The proximal end 12p of the prosthetic valve 12 can abut a distal end 122 of the guide catheter shaft 22 (FIG. 4), which keeps the prosthetic valve 12 in place on the balloon catheter shaft 26 as the delivery apparatus 10 and the prosthetic valve 12 are inserted through an introducer sheath. The proximal end of the valve retaining region 120 is distal to the distal end 78d of the pull wire 78. The prosthetic valve 12 can be delivered in a transfemoral procedure by first inserting an introducer sheath into the femoral artery and pushing the delivery apparatus 10 through the introducer sheath into the patient's vasculature.

After the prosthetic valve 12 is advanced through the narrowest portions of the patient's vasculature (for example, the iliac artery), the prosthetic valve 12 can be moved onto the balloon 28. For example, a convenient location for moving the prosthetic valve onto the balloon is the descending aorta. The prosthetic valve 12 can be moved onto the balloon 28, for example, by holding the handle portion 46 steady (which retains the guide catheter shaft 22 in place), and moving the balloon catheter shaft 26 in the proximal direction relative to the guide catheter shaft 22. As the balloon catheter shaft 26 is moved in the proximal direction, the distal end 122 of the guide catheter shaft 22 pushes against the prosthetic valve 12, allowing the balloon 28 to be moved proximally through the prosthetic valve 12 in order to center the prosthetic valve 12 on the balloon 28, as depicted in FIG. 9. The balloon catheter shaft 26 and/or the guide wire shaft 34 can include one or more radiopaque markers 25 to assist the operator in positioning the prosthetic valve 12 at the desired location on the balloon 28. The balloon catheter shaft 26 can be moved in the proximal direction by simply sliding/pulling the balloon catheter shaft 26 in the proximal direction if the securement mechanism 98 is not engaged to retain the balloon catheter shaft 26. For more precise control of the balloon catheter shaft 26, the securement mechanism 98 can be engaged to retain the balloon catheter shaft 26, in which case the adjustment knob 84 is rotated to effect movement of the balloon catheter shaft 26 and the balloon 28.

As shown in FIG. 5, the delivery apparatus 10 can further include a mounting member 124 secured to the outer surface of the guide wire shaft 34 within the balloon 28. The mounting member 124 helps retain the prosthetic valve 12 in place on the balloon 28 by facilitating the frictional engagement between the prosthetic valve 12 and the outer surface of the balloon 28. The mounting member 124 helps retain the prosthetic valve 12 in place for final positioning of the prosthetic valve 12 at the deployment location, especially when crossing the native leaflets, which typically are calcified and provide resistance against movement of the prosthetic valve 12.

The nose cone 32 has a tapered shape to facilitate atraumatic navigation through the patient's vasculature. For example, the nose cone 32 can taper radially inwardly from a proximal end portion 32p of the nose cone 32 to a distal end 32d (also referred to as a “distal tip”) of the nose cone 32. In the example depicted in FIG. 5, the distal end 37 of the balloon 28 is affixed to the proximal end portion 32p of the nose cone 32.

The nose cone 32 can be connected to a shoulder portion 126 inside the balloon 28 to assist in positioning the prosthetic valve 12. The shoulder portion 126 desirably comprises a tapered member 125 that has a maximum diameter at its proximal end adjacent a distal end 12d of the prosthetic valve 12 (FIG. 9) and tapers in a distal direction toward the nose cone 32.

The shoulder portion 126 serves as a transition section between the nose cone 32 and the prosthetic valve 12 as the prosthetic valve 12 is pushed through the calcified native leaflets by shielding the distal end 12d of the prosthetic valve 12 from contacting the native leaflets. Although FIG. 9 shows the prosthetic valve 12 having a crimped diameter slightly larger than the diameter of the tapered member 125 at its proximal end, the tapered member 125 can have a diameter at its proximal end that is the same as or slightly larger than the diameter of the crimped prosthetic valve, or at least the same as or slightly larger than the diameter of the metal frame of the crimped prosthetic valve. In some examples, the nose cone 32 and the shoulder portion 126 can be constructed as a unitary piece, also referred to as a nose cone assembly.

As shown in FIG. 9, the prosthetic valve 12 can be positioned on the balloon for deployment such that the distal end 12d of the prosthetic valve 12 is slightly spaced from the tapered member 125. When the prosthetic valve 12 is positioned as shown in FIG. 9, the guide catheter shaft 22 can be moved proximally relative to the balloon catheter shaft 26 so that the guide catheter shaft 22 is not covering the inflatable portion of the balloon 28 (and expose the prosthetic valve 12), and therefore will not interfere with inflation of the balloon 28.

As the prosthetic valve 12 is guided through the aortic arch and into the ascending aorta, the curvature of the steerable section 68 can be adjusted (as explained in detail above) to help guide or steer the prosthetic valve 12 through that portion of the vasculature. As the prosthetic valve 12 is moved closer toward the deployment location within the aortic annulus, it becomes increasingly more difficult to control the precise location of the prosthetic valve 12 by pushing or pulling the handle portion 20 due to the curved section of the delivery apparatus. When pushing or pulling the handle portion 20, slack is removed from the curved section of the delivery apparatus before the pushing/pulling force is transferred to the distal end of the delivery apparatus. Consequently, the prosthetic valve may “jump” or move abruptly, making precise positioning of the prosthetic valve difficult.

For more accurate positioning of the prosthetic valve 12 within the aortic annulus, the prosthetic valve 12 is placed as close as possible to its final deployment location (for example, within the aortic annulus such that an inflow end portion of the prosthetic valve 12 is in the left ventricle and an outflow end portion of the prosthetic valve 12 is in the aorta) by pushing/pulling the handle 20, and final positioning of the prosthetic valve 12 is accomplished using the adjustment knob 84. To use the adjustment knob 84, the securement mechanism 98 is placed in its locked position, as described above. Then, the handle 20 is held steady (which retains the guide catheter shaft 22 in place) while rotating the adjustment knob 84 to move the balloon catheter shaft 26, and thus the prosthetic valve 12, in the distal or proximal directions. For example, rotating the knob in a first direction (for example, clockwise), moves the prosthetic valve 12 proximally into the aorta, while rotating the knob in a second, opposite direction (for example, counterclockwise) advances the prosthetic valve 12 distally toward the left ventricle. Advantageously, operation of the adjustment knob 84 is effective to move the prosthetic valve 12 in a precise and controlled manner without sudden, abrupt movements as can happen when pushing or pulling the delivery apparatus 10 for final positioning.

When the prosthetic valve 12 is at the deployment location, the balloon 28 is inflated to expand the prosthetic valve 12 (as depicted in FIG. 11) so as to contact the native annulus. The expanded prosthetic valve becomes anchored within the native aortic annulus by the radial outward force of the valve's frame against the surrounding tissue.

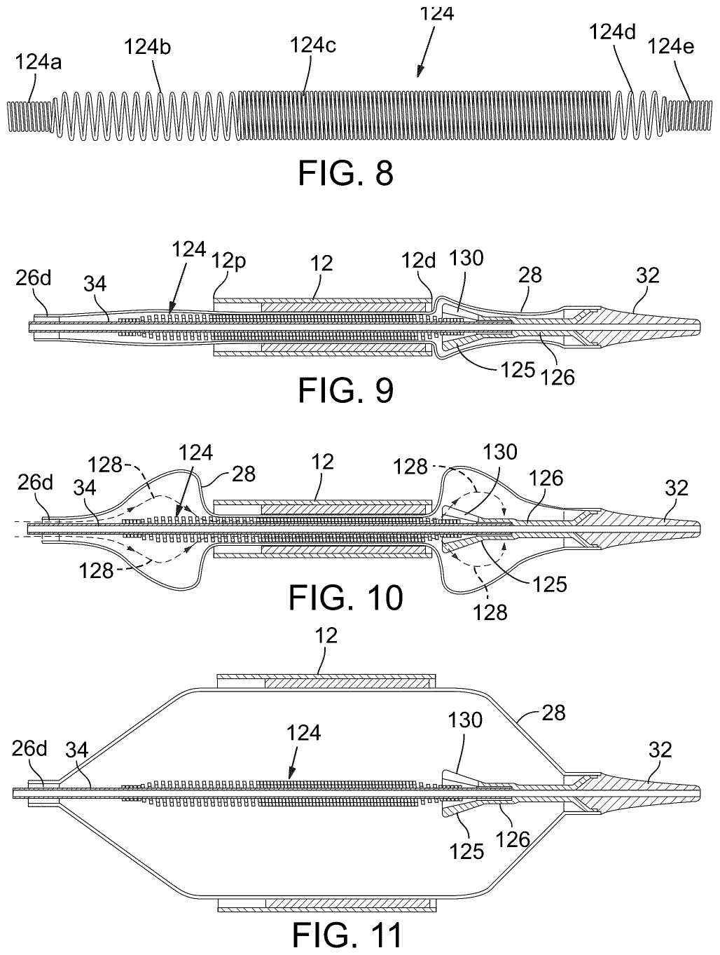

The mounting member 124 within the balloon is configured to allow the inflation fluid (for example, saline) to flow unobstructed from the proximal end 36 of the balloon 28 to the distal end 37 of the balloon 28. As best shown in FIG. 8, for example, the mounting member 124 comprises a coiled wire (for example, a metal coil) having a first section 124a, a second section 124b, a third section 124c, a fourth section 124d, and a fifth section 124e. When the prosthetic valve 12 is positioned on the balloon 28 for deployment, the second section 124b is immediately adjacent the proximal end of the prosthetic valve 12 and the fourth section 124d is immediately adjacent the distal end 12d of the prosthetic valve 12. The first and fifth sections 124a, 124e, respectively, which are at the proximal and distal ends of the mounting member 124, respectively, are secured to the balloon catheter shaft 26. The second, third, and fourth sections 124b, 124c, and 124d, respectively, are relatively larger in diameter than the first and fifth sections and are spaced radially from the outer surface of the balloon catheter shaft 26. As can be seen, the second section 124b and the fourth section 124d can be formed with spaces between adjacent coils. The third section can be formed with smaller spaces (or no spaces) between adjacent coils to maximize the surface area available to retain the prosthetic valve 12 on the balloon 28 during final positioning of the prosthetic valve 12 at the deployment location.

Referring to FIG. 10, the spacing between coils of the second and fourth sections 124b, 124d allows the inflation fluid to flow radially inwardly through the coils of the second section 124b, axially through the lumen of the third section 124c, radially outwardly through the coils of the fourth section 124d, into the distal section of the balloon, in the direction of arrows 128. The tapered member 125 also can be formed with one or more slots 130 that allow the inflation fluid to flow more easily past the tapered member 125 into the distal section of the balloon 28. In the illustrated embodiment, the tapered member 125 has three circumferentially spaced slots 130. Since the inflation fluid can pressurize and inflate the proximal and distal sections of the balloon at substantially the same rate, the balloon 28 can be inflated more evenly for controlled, even expansion of the prosthetic valve 12.

Additional features of the delivery apparatus and some variants of the delivery apparatus are described in U.S. Pat. No. 9,339,384, which is incorporated by reference herein in its entirety.

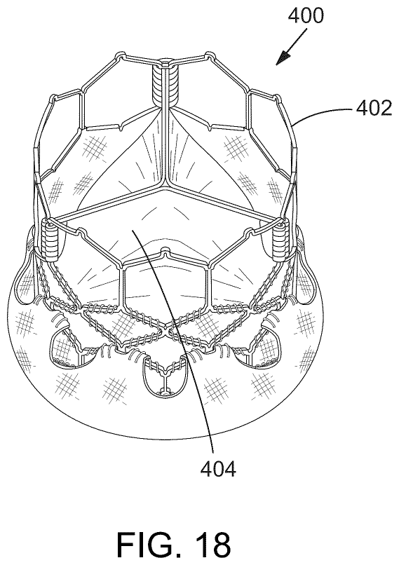

FIG. 18 shows a prosthetic heart valve 400, which can be one specific example of the prosthetic valve 12 described above. As shown, the heart valve 400 comprises a frame, or stent, 402 and a leaflet structure 404 supported by the frame. In some examples, the prosthetic heart valve 400 is adapted to be implanted in the native aortic valve and can be implanted in the body using, for example, the delivery apparatus 10 described above. The prosthetic valve 400 can also be implanted within the body using any of the other delivery apparatuses described herein.

In some examples, the frame 402 comprises a plastically expandable material, which can be metal alloys, polymers, or combinations thereof. Example metal alloys can comprise one or more of the following: nickel, cobalt, chromium, molybdenum, titanium, or other biocompatible metal. In some examples, the frame 402 can comprise stainless steel. In some examples, the frame 402 can comprise cobalt-chromium. In some examples, the frame 402 can comprise nickel-cobalt-chromium. In some examples, the frame 402 comprises a nickel-cobalt-chromium-molybdenum alloy, such as MP35N™ (tradename of SPS Technologies), which is equivalent to UNS R30035 (covered by ASTM F562-02). MP35N™/UNS R30035 comprises 35% nickel, 35% cobalt, 20% chromium, and 10% molybdenum, by weight.

In some examples, the prosthetic valve 12 or 400 can be a self-expandable prosthetic valve with a frame made from a self-expanding material, such as Nitinol. When the prosthetic valve is a self-expanding valve, the balloon of the delivery apparatus can be replaced with a sheath or similar restraining device that retains the prosthetic valve in a radially compressed state for delivery through the body. When the prosthetic valve is at the implantation location, the prosthetic valve can be released from the sheath, and therefore allowed to expand to its functional size. It should be noted that any of the delivery apparatuses disclosed herein can be adapted for use with a self-expanding valve.

### Delivery Apparatus Having a Steerable Guide Wire Shaft

As described above, to implant a prosthetic valve (for example, prosthetic valve 12) in a native heart valve of the patient, the delivery apparatus 10 can be introduced into a vasculature of the patient. The prosthetic valve 12 can be initially retained in a radially compressed configuration on a valve-retaining region 120 of the balloon catheter shaft 26. Once inside the patient's vasculature, the position of the prosthetic valve 12 relative to the balloon 28 can be adjusted such that the prosthetic valve is centered on the balloon 28. When navigating the prosthetic valve through an arched region of the vasculature (for example, the aortic arch), the curvature of the steerable section 68 can be adjusted, for example, by rotating the adjustment knob 56 to tension the pull wire 78. The prosthetic valve can be positioned within or adjacent an annulus of the native heart valve. The prosthetic valve can be positioned within the native annulus using the techniques previously described. Prior to inflating the balloon 28, the guide catheter shaft 22 can be retracted proximally away from the balloon 28 for a sufficient distance so that the guide catheter does not interfere with balloon inflation. This can be accomplished, for example, by holding the Y-connector 24 stationary against the operating table and rotating the adjustment knob 84 in a direction that causes the handle 20 and the guide catheter shaft 22 to move proximally away from the balloon 28. Then, the prosthetic valve can be radially expanded and deployed by inflating the balloon 28.

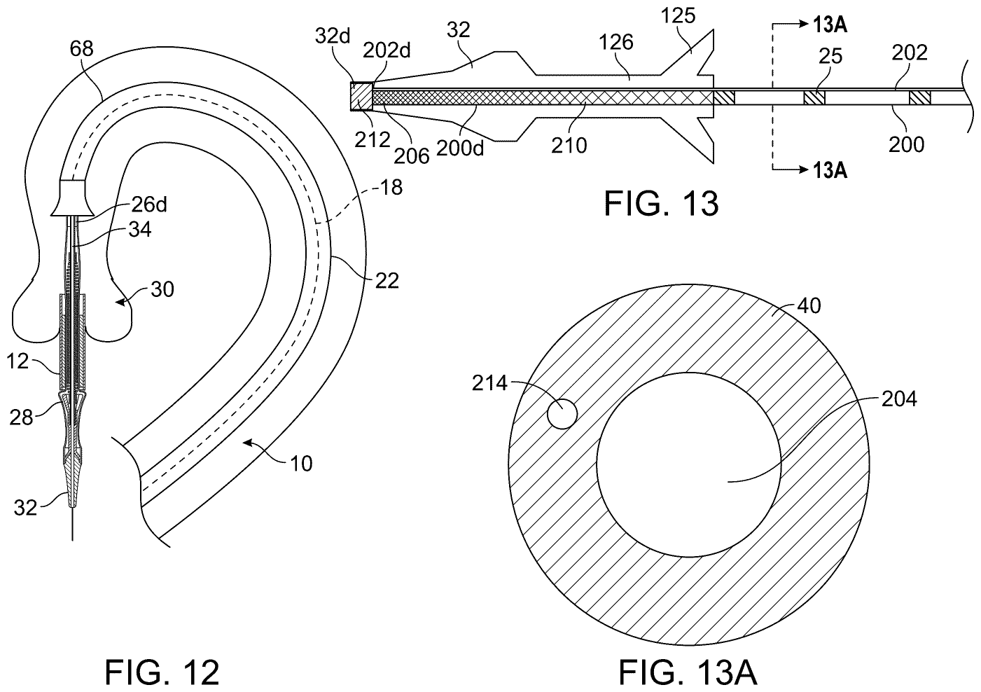

Desirably, when deploying the prosthetic valve, the prosthetic valve should be substantially coaxial with the annulus of the native heart valve so that the prosthetic valve can be evenly expanded and securely anchored within the annulus. In some circumstances, despite the initial position of the heart valve being coaxial with the native annulus, such coaxiality may be disturbed or lost after retracting the guide catheter shaft 22. As shown in FIG. 12, the prosthetic valve 12, mounted on the balloon 28, is aligned substantially coaxially within an aortic annulus 30. Retracting the guide catheter shaft 22 retracts the steerable section 68 farther away from the balloon 28 and the prosthetic valve 12. Without the structural support of the steerable section 68, the distal end portion 26d of the balloon catheter shaft 26 and the distal end portion 34d of the guide wire shaft 34 (where the balloon 28 and the prosthetic valve 12 are mounted) may deflect slightly relative to the steerable section 68. As a result, the prosthetic valve 12 may no longer be coaxial with the aortic annulus 30.

In some examples, an operator can adjust the tension of the pull wire 78 to adjust the curvature of the steerable section 68 to regain coaxiality, that is, to realign the central (longitudinal) axis of the prosthetic valve 12 with the central axis of the aortic annulus 30. However, such manipulation can be difficult. Because the distal end of the pull wire 78 ends at the steerable section 68, the tensile force applied to the pull wire 78 may only be partially imparted to the distal end portion 26d of the balloon catheter shaft 26 and the distal end portion 34d of the guide wire shaft 34 to adjust the orientation of the prosthetic valve 12.

In some examples, the orientation of the distal end portion of the balloon catheter shaft 26 and the distal end portion of the guide wire shaft 34 can be independently adjusted after retracting the guide catheter shaft 22, thereby facilitating fine tune position of the prosthetic valve 12 to regain coaxiality with the native annulus. This can be helpful prior to balloon inflation in which coaxial alignment of the prosthetic valve 12 with the native annulus is desirable for repeatable valve expansions. This can be achieved, for example, by manipulating a separate pull wire connected to the nose cone 32 and attached to an actuation mechanism in the Y-connector 24.

FIG. 13 depicts a nose cone 32 mounted on a guide wire shaft 200 having a pull wire 202, according to one example. As noted above, the nose cone 32 and the shoulder portion 126 can be constructed as a unitary piece, which can be referred to as a nose cone assembly. In some examples, the nose cone 32 and the shoulder portion 126 can be separate components and may be axially spaced from each other along the guide wire shaft 200. In some examples, the guide wire shaft 200 does not have a shoulder portion 126.

The guide wire shaft 200 can replace the guide wire shaft 34 of the delivery apparatus 10. For example, the guide wire shaft 200 can extend from the Y-connector 24 of the balloon catheter 16 through the lumen 23 of the balloon catheter shaft 26 and the balloon 28. Similarly, the guide wire shaft 200 can also have a guide wire lumen 204 (FIG. 13A) configured to receive the guide wire 18.

In contrast to the guide wire shaft 34, which is bound to and terminates at the shoulder portion 126 (FIG. 5), the guide wire shaft 200 extends through the shoulder portion 126 and terminates at or near the distal end 32d of the nose cone 32 (FIG. 13). In one example, a distal end 206 of the guide wire shaft 200 can extend to the distal end 32d of the nose cone 32. This can facilitate further control of the distal end of the delivery apparatus for coaxial alignment of the prosthetic valve 12 with the native annulus prior to balloon inflation.

The guide wire shaft 200 can also have a braided layer 210 configured to impart desired flexibility and stiffness to different parts of the guide wire shaft 200. For example, the braided layer 210 can extend into the lumen 33 of the nose cone 32. In certain examples, the braided layer 210 can also extend proximally from the nose cone 32 along the entire length or substantially the entire length of the shaft 200. The braided layer 210 can extend over one or more polymeric layers of the shaft 200. In some examples, the braided layers can be constructed from braided metal wires (for example, stainless steel wires, Nitinol wires, etc.).

In certain examples, the braided layer 210 has a braid density that is greater along a distal end portion 200d of the guide wire shaft 200 than along a proximal end portion 200p of the guide wire shaft 200. For example, as depicted in FIG. 13, the section of the braided layer 210 in the nose cone 32 has a higher braid density than the section of the braided layer 210 in the shoulder portion 126. In one example, the braid density can progressively decrease from the distal end 32d of the nose cone 32 to the tapered member 125 of the shoulder portion 126. Generally, a higher braid density can provide a greater curvature along the distal end portion of the guide wire shaft 200 when tension in the pull wire 202 is increased.

In some examples, the section of the braided layer in the nose cone 32 comprises a 2 under 2, over 2 braid and the section of the braided layer in the shoulder portion 126 comprises a 1 under 2, over 2 braid.

The pull wire 202 can extend longitudinally along the guide wire shaft 200. For example, as depicted in FIG. 13A, the guide wire shaft 200 can have a side lumen 214 radially offset from the guide wire lumen 204. The pull wire 202 can extend through the side lumen 214 at least partially along the length of the pull wire 202. In some examples, the pull wire 202 can be covered by and/or embedded within the braided layer 210, at least along the section of the braided layer that extends within the shoulder portion 126 and the nose cone 32.

A distal end 202d of the pull wire 202 can be fixed relative to the guide wire shaft 200 at a location that is distal to the distal end 37 of the balloon 28. In one example, as depicted in FIG. 13, the distal end 202d of the pull wire 202 is fixedly connected to a ring 212 (also referred to as a “pull ring”) located at the distal end 32d of the nose cone 32. Thus, by adjusting the tension in the pull wire 202, a curvature of the distal end portion 200d of the guide wire shaft 200 and the nose cone 32 can be adjusted. Because the guide wire shaft 200 extends through the lumen 23 of the balloon catheter shaft 26, bending the distal end portion 200d of the guide wire shaft 200 can in turn impart a force to, and change the orientation of, the distal end portion 26d of the balloon catheter shaft 26. As a result, alignment of the balloon 28 and the prosthetic valve 12 mounted thereof can also be adjusted.

Tensioning the pull wire 202 can be performed separately from tensioning the pull wire 78. As a result, the curvature of the nose cone 32 and the distal end portion 200d of the guide wire shaft 200 can be adjusted independently from adjusting the curvature of the steerable section 68 of guide catheter shaft 22. Because the distal end 202d of the pull wire 202 is distal to the balloon 28 and the prosthetic valve 12 (in contrast to the distal end 78d of the pull wire 78 which is proximal to the balloon 28 and the prosthetic valve 12), tensioning the pull wire 202 can be more effective to change the orientation of the balloon 28 and the prosthetic valve 12 relative to the native annulus than tensioning the pull wire 78.

Thus, after retracting the guide catheter shaft 22 and before inflating the balloon 28, if it is found that the prosthetic valve 12 is not coaxial with the native annulus, the operator can adjust the tension of the pull wire 202 to change the curvature of the nose cone 32 and the distal end portion 200d of the guide wire shaft 200 until the axial axis of the prosthetic valve 12 is substantially aligned with the central axis of the native annulus.

In some examples, tensioning the pull wire 202 mainly increases the curvature of the nose cone 32 (and the distal end portion 200d of the guide wire shaft 200 embedded therein). In some examples, the curvature of the shoulder portion 126 may also be slightly increased but is less than the curvature of the nose cone 32 because the braided layer of the guide wire shaft 200 has a smaller density in the shoulder portion 126 than in the nose cone 32. Further, tensioning the pull wire 202 causes little or no perceptible bending of the balloon 28 and the prosthetic valve 12 mounted thereof. The guide wire shaft 200 has sufficient structural integrity to be robust to this tensioning via the pull wire 202. This can be accomplished, for example, by using a sufficiently low braid density for the portion of the guide wire shaft 200 that extends through the balloon 28. In other words, the relatively high braid density of the guide wire shaft 200 is limited to the region inside the nose cone 32, which is distal to the balloon 28. By limiting the bending to the nose cone 32 and the distal end portion 200d of the guide wire shaft 200 that are distal to the balloon 28, the operator can adjust the alignment of the prosthetic valve 12 while avoiding bending the prosthetic valve 12. The axial length of the nose cone 32 (measured from its proximal end 32p to the distal end 32d; see FIG. 5) can be configured to support a predefined range of curvature. In some examples, the axial length of the nose cone 32 can range from 5.00 mm to 7.5 mm, or from 5.0 mm to 10.0 mm, inclusive. It should be noted that the additional deflection can occur beginning at the tapered member 125 and continue to the distal end 32d of the nose cone 32d. In some examples, the axial length of this region (that is, from 125 to 32d) can range from 32.0 mm to 40.0 mm, inclusive.

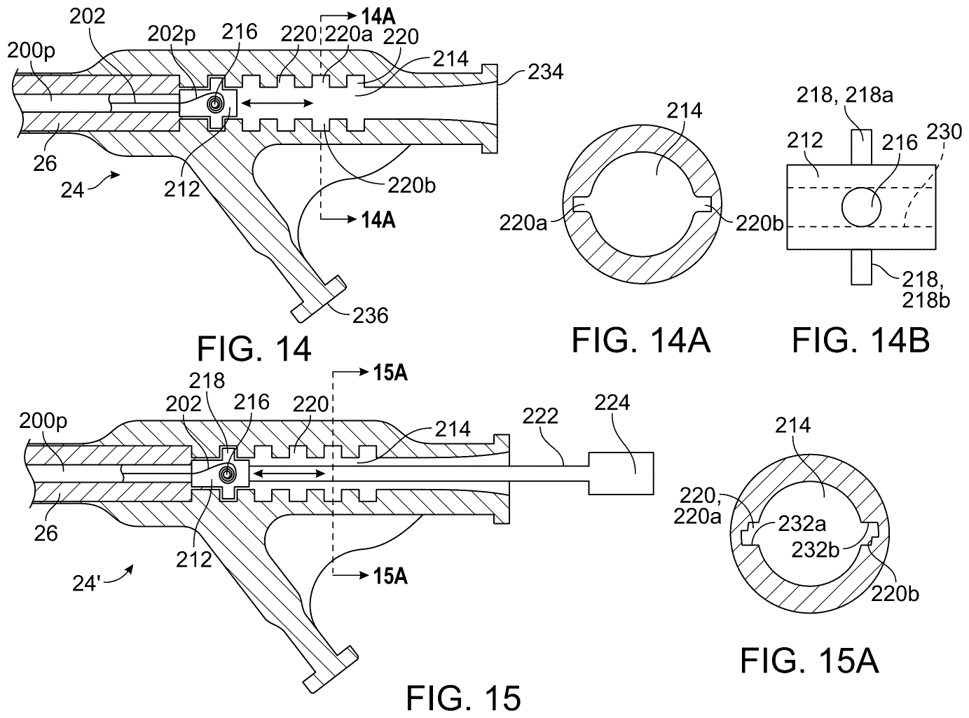

Tensioning the pull wire 202 can be accomplished by actuating an actuator or actuation mechanism connected to a proximal end 202p of the pull wire 202. For example, FIG. 14 shows that the proximal end 202p of the pull wire 202 can be connected to an actuator in the form of a sliding member 212 housed within the Y-connector 24, which is positioned proximal to the handle 20. The sliding member 212 is configured to move the pull wire 202 axially relative to the handle 20, thereby adjusting the curvature of the nose cone 32 and the distal end portion 200d of the guide wire shaft 200 through a tensile force applied to the distal end 202d of the pull wire 202. The Y-connector 24 can include a locking mechanism configured to lock an axial position of the sliding member 212 and the proximal end 202p of the pull wire 202. Thus, when a desired curvature of the nose cone 32 is achieved, the operator can fix the curvature of the nose cone 32 by fixing the axial position of the sliding member 212 and the proximal end 202p of the pull wire 202. The Y-connector 24 can have a proximal port 234 through which a guide wire 18 can extend and a side port 236 through which an inflation fluid for inflating the balloon 28 can be injected.

As in FIG. 14, the sliding member 212 is axially movable within a channel 214 of the Y-connector 24. The proximal end 202p of the pull wire 202 can be connected to and/or wrapped around a post 216 of the sliding member 212. Thus, moving the sliding member 212 (along the channel 214) in a proximal direction relative to the Y-connector 24 can tighten or increase tension of the pull wire 202 and pull the distal end 32d of nose cone 32 back toward the Y-connector 24 and reduce its radius of curvature, thereby bending the nose cone 32 and the distal end portion 200d of the guide wire shaft 200. Conversely, moving the sliding member 212 (along the channel 214) in a distal direction relative to the Y-connector 24 can reduce the tension of the pull wire 202 and increase the radius of curvature for the nose cone 32. The sliding member 212 can include a lumen 230 that allows a guide wire 18 and a liquid (for example, an inflation fluid) to pass through the sliding member 212.

As depicted in FIGS. 14 and 14A, the locking mechanism can comprise a plurality of recesses 220 distributed along the channel 214 of the adaptor 24. The recesses 220 are configured to receive one or more protruding pins 218 of the sliding member 212. In one example, the recesses 220 can be arranged in pairs of recesses 220a, 220b located on diametrically opposite sides of the channel 214, and sliding member 212 can have two opposing pins or posts 218a, 218b that can be positioned in respective recesses 220a, 220b of a selected pair of recesses. When the pins 218 are received in the recesses 220, the sliding member 212 is in a locked configuration, that is, axial movement of the sliding member 212 within the channel 214 is disabled. When the pins 218 are moved out of the recesses 220, the sliding member 212 is in an unlocked configuration, that is, axial movement of the sliding member 212 relative to the channel 214 is enabled.

To adjust the position of the sliding member 212 (and therefore the tension in the pull wire 202), the pins 218 are removed from the recesses, such as by moving the sliding member 212 laterally out of the channel 214 (for example, in a direction that is out of or into the page as shown in FIG. 14), then moving the sliding member 212 in an axial direction (proximally or distally), and then positioning the sliding member back in the channel with the pins 218 positioned in another pair of recesses. To permit access to the sliding member in the channel, the Y-connector 24 can be configured to be openable. For example, the Y-connector 24 can have a clam-shell configuration that allows two portions or halves of the Y-connector to be moved between an open configuration (to expose and allow access to the sliding member 212 and a closed configuration (to enclose and prevent access to the sliding member 212).

Moving the sliding member 212 in a proximal direction (for example, toward the proximal port 234) increases tension in the pull wire 202 and increases the curvature of the nose cone 32, while moving the sliding member 212 in a distal direction (for example, toward the handle 20) decreases tension in the pull wire 202 and reduces the curvature of the nose cone 32. During an implantation procedure, the sliding member 212 initially can be positioned at the distal-most pair of recesses 220a, 220b until the shaft 22 is retracted and the prosthetic valve 12 is positioned within the native annulus. If retracting the shaft 22 causes the prosthetic valve to move out of coaxial alignment with respect to the native annulus, the tension in the pull wire 202 can be adjusted by moving the sliding member 212 in the proximal direction until a desired alignment of the prosthetic valve is achieved. The curvature of the nose cone 32 and therefore the position and orientation of the prosthetic valve relative to the native annulus can be fixed by positioning the pins 218 of the sliding member 212 in the recesses 220 at the location of the sliding member within the channel. Thereafter, the balloon 28 can be inflated to deploy the prosthetic valve 12 into engagement with the native annulus.

Although the channel 214 in the illustrated example includes five pairs of recesses 220a, 220b, there can be a greater or few number of pairs of recesses. The number of recesses 220, the position of recesses 220 along the channel 214, and/or the spacing between adjacent pairs of recesses 220 can vary to create different tensions of the pull wire 202 and different curvatures at the nose cone 32. In some examples, the pairs of recesses 220 are positioned along a 3-cm length of the channel 214. In one specific example, there are seven pairs of recesses 220a, 220b distributed along a 3-cm length of the channel 214, with a distance about 5 mm between each two adjacent pairs of recesses 220a, 220b. In other examples, a greater or fewer number of recesses with the same or different inter-recess distances can be used.

FIG. 15 depicts another Y-connector 24′ which is similar to the Y-connector 24 except that the sliding member 212 is connected to a handle 224 located outside the Y-connector 24′ by a handle shaft 222. Another difference is that the channel 214 is formed with opposed recesses 232a, 232b that extend longitudinally along the channel 214 between adjacent recesses 220a, 220b, respectively. To adjust the position of the sliding member 212 along the channel, the operator can rotate the handle 224 to rotate the sliding member 212, which rotates the pins 218a, 218b out of their respective recesses 220a, 220b and into respective recesses 232a, 232b. With the pins in the recesses 232a, 232b, the handle 224 can be used to push or pull the sliding member 212 within the channel 214 to adjust the tension in the pull wire 220. After the sliding member 212 is moved to a new location at a different pair of recesses 220a, 220b, the handle 224 can be rotated in the opposite direction to rotate the pins 218a, 218b, into the recesses 220a, 220b, thereby locking the sliding member 212 at its new location along the length of the channel 214.

Although FIGS. 14-15 show specific examples of actuation and locking mechanisms, it is to be understood that the pull wire 202 can be actuated and/or locked by other mechanisms. For example, the flex activating mechanism 50 of the handle 20 (including knob 56) for adjusting the tension of the pull wire 78 described above can be used for adjusting the tension in the pull wire 202. In some examples, the Y-connector 24 can be configured to have a flex activating mechanism 50 with a rotatable knob 56 (or a similar mechanism), wherein the knob is rotatable to move the sliding member 212 within the channel 214 of the Y-connector 24. In some examples, the end portion of the guide wire shaft 200 can be coupled to a separate handle, which can be proximal or distal to the Y-connector 24. The separate handle can include an adjustment mechanism, such as the flex activating mechanism 50, that is configured to move the sliding member 212 within the separate handle.

### Additional Examples of Steerable Nose Cones

As described above, to implant a prosthetic device (for example, prosthetic valve 12) in a patient, the prosthetic device can be mounted on a delivery apparatus (for example, delivery apparatus 10), which can be introduced into the patient's vasculature. When navigating through an arched region of the vasculature (for example, the aortic arch), the curvature of the delivery apparatus can be adjusted by tensioning one or more pull wires. For example, as described above, tensioning the pull wire 78 can adjust the curvature of the steerable section 68 of the guide catheter shaft 22. Additionally, tensioning the pull wire 202 can adjust the curvature of the distal end portion 200d of the guide wire shaft 200 and the nose cone 32.

In the examples described above, pull wires extending along the shafts are substantially parallel to the central or longitudinal axis of those shafts, which are coaxial. For example, both the pull wire 78 extending along the guide catheter shaft 22 and the pull wire 202 extending along the guide wire shaft 200 are substantially parallel to the longitudinal axis of the guide wire shaft 200. In some cases, a relatively large tension force must be applied in order to provide a sufficient torque to bend the corresponding portions of the delivery apparatus. For example, to increase the curvature of the distal end portion 200d of the guide wire shaft 200 and the nose cone 32, an increased tension force must be applied to the pull wire 202. Since the pull wire 202 is substantially parallel to the longitudinal axis of the guide wire shaft 200, the required tension force can be quite large in order to achieve a desired rotational torque in order to flex the nose cone 32 (and the distal end portion 200d of the guide wire shaft 200 embedded therein).

Further, tensioning the pull wire 202 not only can bend the nose cone 32 and the distal end portion 200d of the guide wire shaft 200, but also may cause flexion of other portions of the delivery apparatus that are located proximal to the nose cone 32 (for example, the shoulder portion 126 and/or the valve retaining region 120, together with the portions of the guide wire shaft 200 extending therethrough). Bending a longer portion of the delivery apparatus can require an even larger tension force to be applied to the pull wire 202. Moreover, the resulting radius of curvature of the curved portion of the guide wire shaft 200 can be relatively large (corresponding to a relatively small central angle of the curved portion of the guide wire shaft 200), which can make it difficult to steer the delivery apparatus through sharp-angled curved portion of the vasculature.

Many of the challenges described above can be addressed by using bendable nose cones as described below. The bendable nose cones described below can replace the nose cone 32 of the delivery apparatus 10.

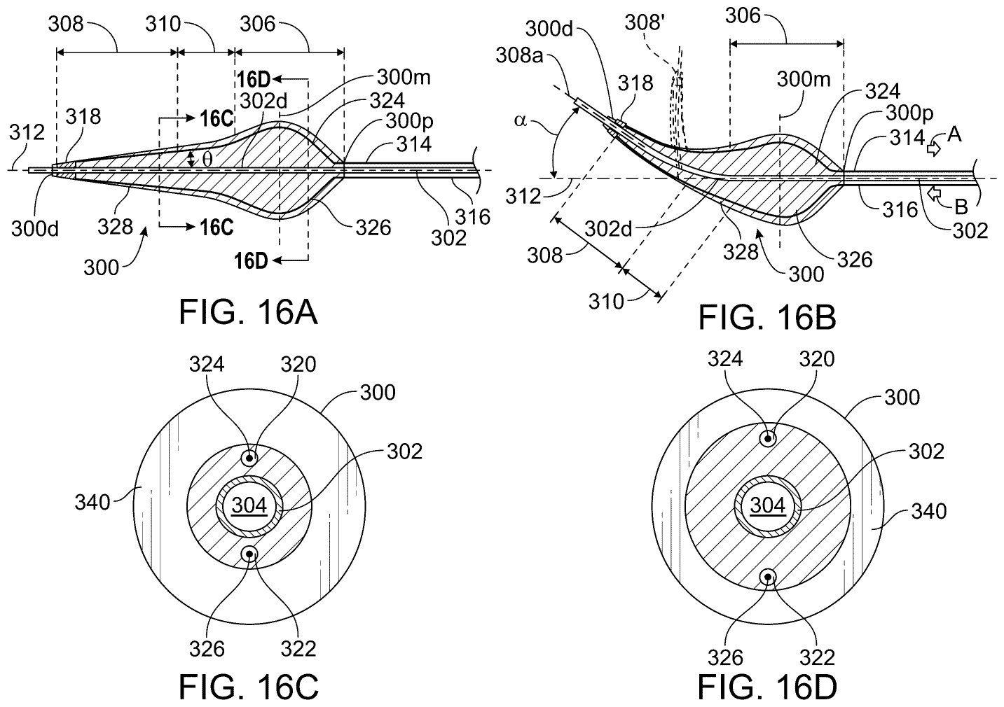

FIGS. 16A-16D depicts a bendable nose cone 300, according to one example. The nose cone 300 is mounted on (and coaxial with) a distal end portion 302d of a guide wire shaft 302 (similar to the guide wire shaft 34), which extends distally from a handle (for example, handle 20). Similarly, the guide wire shaft 302 has a guide wire lumen 304 configured to receive a guide wire (for example, guide wire 18).

As shown, the nose cone 300 has a proximal portion 306, a distal portion 308, and a flexible or intermediate portion 310 located between the proximal portion 306 and the distal portion 308. In some examples, the flexible portion 310 can be deemed part of the distal portion 308.

The largest diameter of the nose cone 300 is located along the proximal portion 306, marked by the dotted line 300m. The part of the nose cone distal to the line 300m (including the distal portion 308 and the flexible portion 310) has a tapered shape with a progressively reduced diameter in the distal direction until reaching a distal end 300d of the nose cone 300. The part of the nose cone proximal to the line 300m can also taper radially inwardly until reaching a proximal end 300p of the nose cone 300. In some examples, the nose cone 300 has a larger diameter at the proximal end 300p than at the distal end 300d. For example, the diameter of the nose cone at the distal end 300d can be about 1.8 mm, and the diameter of the nose cone at the proximal end 300p can range from 2 mm to 5 mm, inclusive. In some examples, the nose cone 300 can have about the same diameter at the proximal end 300p and the distal end 300d.

The nose cone 300 is configured to be movable between a straightened state (as depicted in FIG. 16A) and a flexed state (as depicted in FIG. 16B). In the straightened state, the nose cone 300 is substantially straight. That is, both the proximal portion 306 and the distal portion 308 of the nose cone are substantially aligned with a longitudinal axis 312 of the guide wire shaft 302. Correspondingly, the distal end portion 302d of the guide wire shaft 302 remains substantially straight and aligned with the longitudinal axis 312. In the flexed state, the proximal portion 306 of the nose cone remains substantially straight and aligned with the longitudinal axis 312. However, the distal portion 308 is rotated or flexed relative to the proximal portion 306 so that the distal end 300d of the nose cone moves in an angular direction toward the handle. As a result, a central axis 308a of the distal portion 308 extends away from the longitudinal axis 312 so as to form a bend angle (α) between the two axes 308a and 312. Accordingly, the distal end portion 302d of the guide wire shaft 302 within the nose cone has a curved shape.

Generally, a larger bend angle α can be achieved by increasing the total axial length of the distal portion 308 and the flexible portion 310. In some examples, the total axial length of the distal portion 308 and the flexible portion 310 can range from 10 mm and 20 mm (for example, about 15 mm). The proximal portion 308, which generally has a larger diameter than the distal portion 306, is generally non-bendable in certain examples. In some examples, the axial length of the proximal potion 306 can range from 3 mm to 10 mm, or from 5 mm to 8 mm.

In some examples, the maximum bend angle α can be acute. In some examples, the maximum bend angle α can be 90 degrees, that is, the central axis 308a can be perpendicular to the longitudinal axis 312, as indicated by the dashed distal portion 308′ in FIG. 16B. In some examples, the maximum bend angle α can be greater than 90 degrees (for example, up to 120 degrees or 150 degrees, etc.).

Allowing the bend angle α to be up to 90 degrees (or more) can facilitate easier passage of the delivery apparatus through curved portions of the vasculature, and can also provide improved maneuverability through local obstacles when required. For example, in some circumstances, when inserting the delivery apparatus into the heart, crossing a stenosed native aortic valve may be difficult, for example, due to calcified leaflets of the native aortic valve. In such cases, bending the distal portion 308 relative to the proximal portion 306 to achieve a sufficiently large bend angle α may facilitate passage of the nose cone 300 through the native leaflets alone the guide wire.

In some examples, bending of the nose cone 300 can be limited to the flexible portion 310 so that the distal portion 308 remains substantially straight when the nose cone 300 is in the flexed state. In some examples, the distal portion 308 itself can have a curved shape when the nose cone 300 is in the flexed state.

The flexibility of the nose cone 300 can vary along its length to allow rotating or bending of the distal portion 308 but not the proximal portion 306 of the nose cone. Specifically, the distal portion 308 and/or the flexible portion 310 can have a lower flexural modulus (that is, more flexible) than the proximal portion 306. For example, the nose cone 300 can be made from a polyether block amide (PEBA) thermoplastic elastomer, such as Pebax, that can have a relatively harder proximal portion (for example, Pebax having a durometer of 45D) and a relatively softer distal portion (for example, Pebax having a durometer of 35D or 30D). In some examples, variations in flexibility can be achieved by varying the structure, geometry, and/or materials used in different parts of the nose cone 300. For example, the nose cone 300 can comprise different materials to provide different flexibility along its axial length. In some examples, the distal portion 308 can comprise a first material and the proximal portion 306 can comprise a second material that is stiffer (less flexible) than the first material. In some examples, the flexible portion 310 can comprise a first material, and both the distal portion 308 and the proximal portion 306 can comprise a second material that is stiffer (less flexible) than the first material. As a result, when a tension force is applied to the distal end 300d of the nose cone, the distal portion 308 can rotate relative to the proximal portion 306 (for example, by bending the flexible portion 310) while the proximal portion 306 can remain substantially coaxial with the longitudinal axis 312.

As described herein, moving the nose cone 300 between the straightened state and the flexed state can be actuated by adjusting the tension in one or more pull wires connected to the nose cone 300. In the example depicted in FIGS. 16A and 16B, two pull wires 314, 316 are shown for the nose cone 300. In some examples, the number of pull wires can be only one or more than two (for example, three, four, etc.).

The proximal end of the pull wires 314, 316 can be connected to the handle (for example, handle 20). In some examples, the tension in each pull wire 314 or 316 can be independently adjusted (for example, by rotating a knob similar to knob 56). In some examples, the tension in the two pull wires 314, 316 can be adjusted simultaneously such that increasing the tension in the pull wire 314 can simultaneously decrease the tension in the pull wire 316, or vice versa. In some examples, the pull wires 314, 316 can be connected to a locking mechanism configured to restrict axial movement of the pull wires 314, 316 relative to the guide wire shaft 302.

The pull wires 314 and 316 extend longitudinally along the guide wire shaft 302 and into the nose cone 300. Specifically, the portion of each pull wire 314 or 316 between the handle and the nose cone 300 is substantially parallel to the longitudinal axis 312. In the example depicted in FIGS. 16C and 16D, the pull wires 314 and 316 have respective distal end portions 324, 326 that are completed embedded within the nose cone 300. The distal ends of the pull wires 314, 316 are connected to the distal end 300d of the nose cone. For example, a ring 318 can be disposed at the distal end 300d, and the distal ends of the pull wires 314 and 316 can be fixedly connected to the ring 318. The ring 318 can form the distal tip of the nose cone 300 as shown. Alternatively, the ring 318 can be embedded within the material forming the distal portion 308 of the nose cone.

As shown in FIGS. 16A-16B, the distal end portions 324, 326 of the pull wires are nonparallel to the distal end portion 302d of the guide wire shaft. In other words, the radial distance between the distal end portions 324, 326 of the pull wires and the distal end portion 302d of the guide wire shaft can vary in the axial direction (for example, gradually increase from the proximal end 300p of the nose cone to the line 300m, and then gradually decrease from the line 300m to the distal end 300d of the nose cone).

As depicted in FIGS. 16C-16D, the distal end portions 324, 326 of the pull wires can extend through respective guide wire lumens or channels 320, 322, respectively, within the nose cone 300. The channel 320, 322 and the distal end portions 324, 326 of the pull wires can extend alongside an axial profile 328 of the nose cone 300, starting from the proximal end 300p and ending at the distal end 300d of the nose cone. Stated differently, the channels 320, 322 and the distal end portions 324, 326 of the pull wires extending therethrough can extend along respective paths that conform to the axial profile 328 of the nose cone 300. The axial profile 328 defines the curvature of an outer surface 340 of the nose cone 300 taken along its longitudinal cross-section (as shown in FIGS. 16C-16D). In some examples, the radial distance between the channel 320 (or 322) and the adjacent outer surface 340 of the nose cone remains substantially constant throughout the nose cone. In some examples, the radial distance between the channel 320 (or 322) and the adjacent outer surface 340 of the nose cone can vary in different parts of the nose cone. For instance, the radial distance between the channel 320 (or 322) and the adjacent outer surface 340 can be larger in the proximal portion 306 than in the distal portion 308.

As shown in FIGS. 16A-16B, the two distal end portions 324, 326 of the pull wires are in mirrored positions relative to the distal end portion 302d of the guide wire shaft, that is, the distal end portions 324, 326 can be on diametrically opposite sides of the distal end portion 302d of the guide wire shaft. When more than two pull wires extend into the nose cone 300, the distal end portions of these pull wires can be configured to be rotationally symmetric about the distal end portion 302d of the guide wire shaft (for example, the distal end portions of four pull wires can be arranged to be 90 degrees apart from one another).

Adjusting the tensions in the pull wires 314, 316 can move the nose cone 300 from the straightened state to the flexed state, or vice versa. For example, as illustrated in FIG. 16B, pulling the pull wire 314 in the proximal direction (as indicated by the block arrow A) while allowing the pull wire 316 to freely extend in the distal direction (as indicated by the block arrow B) can rotate or bend the distal portion 308 in a first direction (for example, in an upward direction in FIG. 16B) relative to the proximal portion 306. Conversely, pulling the pull wire 316 in the proximal direction while allowing the pull wire 314 to freely extend in the distal direction can rotate or bend the distal portion 308 in a second direction, opposite the first direction, relative to the proximal portion 306. In both cases, the proximal portion 306 of the nose cone 300 can remain substantially straight and coaxial with the longitudinal axis 312.

When more than two pull wires extend into the nose cone 300, selected pull wires can be tensioned (for example, pulling in the proximal direction) and/or un-tensioned (for example, allowing to freely extend in the distal direction) so as to rotate or bend the distal portion 308 of the nose cone in a desired angular direction while keeping the proximal portion 306 of the nose cone substantially straight and coaxial with the longitudinal axis 312.

When there is a single pull wire (for example, pull wire 314 or 316) extending into the nose cone 300, tensioning such pull wire can rotate or bend the distal portion 308 relative to the proximal portion 306 in an angular direction within a plane defined by the pull wire and the longitudinal axis 312. To rotate or bend the distal portion 308 in another angular direction, the guide wire shaft 302 (along with the pull wire) can be first rotated about the longitudinal axis 312 a desired amount such that the pull wire is angularly spaced from its initial position, after which the pull wire can be tensioned.

Compared to the example depicted in FIG. 13 where the pull wire 202 extends longitudinally along (and located adjacent) the guide wire shaft 200 throughout the entire length of the guide wire shaft 200, the examples of FIGS. 16A-16B show that the radial distance between the distal end portions 324, 326 of the pull wires and the distal end portion 302d of the guide wire shaft can be much larger (only limited by the diameter of the nose cone), especially in the proximal portion 306 of the nose cone. Such increased radial distance results in an increased pulling angle (θ) between the distal end portion 324 (or 326) of the pull wire and the distal end portion 302d of the guide wire shaft (compared to the example of FIG. 13 where the pulling angle between the pull wire 202 and the guide wire shaft 200 is close to zero). When applying a tension force to the pull wire 314 (or 316), increasing the pulling angle θ can impart a larger torque to rotate the distal end portion 308 of the nose cone. As a result, in order to apply a sufficient torque to bend the nose cone 300 to a desired bend angle α, a smaller amount of tension force can be applied to the pull wire 314 (or 316).

Furthermore, by limiting the bending of the nose cone 300 to the distal portion 308 and/or the transition portion 310 while keeping the proximal portion 306 substantially straight, the required tension force applied to the pull wire 314 (or 316) can be further reduced compared to the example of FIG. 13, in which tensioning the pull wire 202 can bend the entire nose cone 32 and the guide wire shaft 200 extending therethrough.

In the example of FIGS. 16A-16D, the portions of the pull wires 314, 316 proximal to the nose cone are shown extending outside and alongside the guide wire shaft 302. In some examples, the portions of the pull wires 314, 316 proximal to the nose cone can extend through the guide wire shaft 302, such as through respective channels or guide wire lumens of the shaft 302 (for example, respective lumens similar to lumen 214 of FIG. 13A).

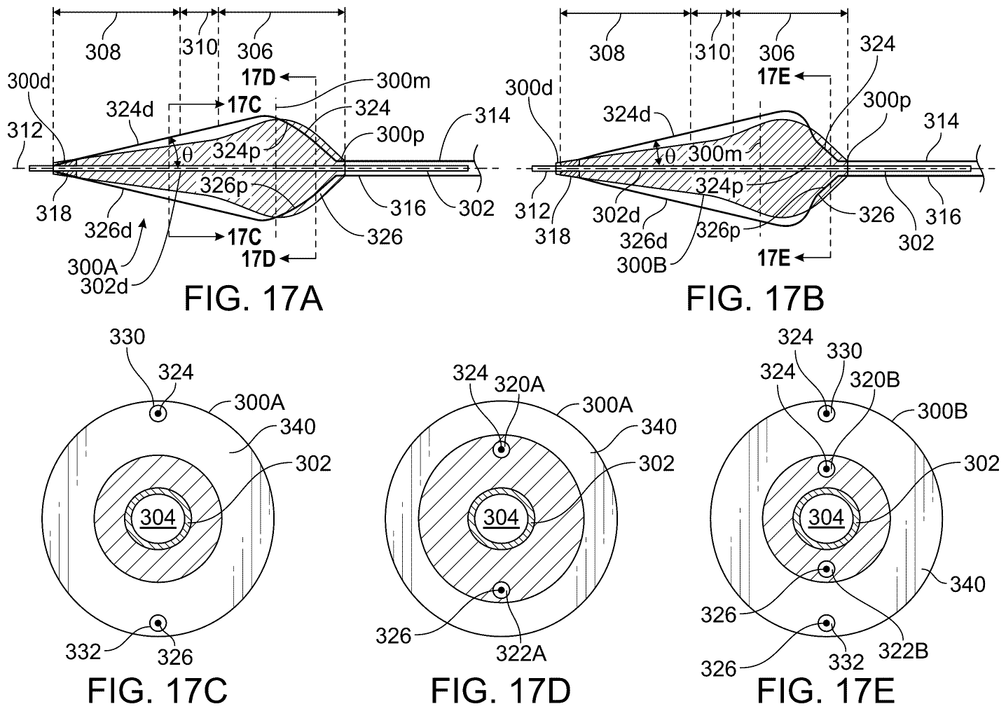

FIGS. 17A-17B depict two bendable nose cones 300A and 300B, according to some examples. Similarly, the nose cone 300A or 300B is mounted on (and coaxial with) the distal end portion 302d of the guide wire shaft 302, and one or more pull wires extend longitudinally along the guide wire shaft 302 and into the nose cone 300A or 300B. Although two pull wires 314, 316 are shown in FIGS. 17A-17B, in some examples, only a single pull wire or more than two pull wires can extend into the nose cone 300A or 300B. The nose cone 300A or 300B can be the same as the nose cone 300 except for having different routes to receive the distal end portions 324, 326 of the pull wires. In contrast to the nose cone 300 in which the channels 320, 322 extend between the proximal end 300p and the distal end 300d of the nose cone, the nose cone 300A or 300B has channels which only extend through the proximal portion 306 of the nose cone.

As shown in FIGS. 17A and 17C-17D, the nose cone 300A has channels 320A and 322A extending through the proximal portion 306 of the nose cone. Specifically, the channels 320A, 322A have respective side openings 330, 332 on the outer surface 340 of the nose cone. The channel 320A, 322A can extend alongside the axial profile of the proximal portion 306, starting from the proximal end 300p and ending at the side openings 330, 332. In FIG. 17A, the side openings 330, 332 are adjacent to the line 300m, which marks the largest diameter of the nose cone 300A. Thus, the distal end portions 324, 326 of the pull wires can extend through respective channels 320A, 322A, exit the nose cone at corresponding side openings 330, 332, and then extend alongside the outer surface of the nose cone. Similar to the example of FIGS. 16A-16D, the distal end portions 324, 326 of the pull wires can be connected to the distal end 300d (for example, at the ring 318) of the nose cone 300A. As a result, while proximal segments 324p, 326p of the distal end portions 324, 326 of the pull wires are embedded with the nose cone 300A, distal segments 324d, 326d of the distal end portions 324, 326 of the pull wires extend outside the nose cone 300A.

Similarly, as shown in FIGS. 17B and 17E, the nose cone 300B has channels 320B and 322B extending alongside the axial profile of the proximal portion 306, starting from the proximal end 300p and ending at respective side openings 330, 332 on the outer surface 340 of the nose cone. In this example, the side openings 330, 332 are proximal to the line 300m. Thus, the distal end portions 324, 326 of the pull wires can extend through the respective channels 320B, 322B, exit the nose cone at corresponding side openings 330, 332, and then be connected to the distal end 300d of the nose cone 300B. As a result, while proximal segments 324p, 326p of the distal end portions 324, 326 of the pull wires are embedded with the nose cone 300B, distal segments 324d, 326d of the distal end portions 324, 326 of the pull wires extend outside the nose cone 300B.

In the examples of FIGS. 17A-17B, the two distal end portions 324, 326 of the pull wires are in mirrored positions relative to the distal end portion 302d of the guide wire shaft. When more than two pull wires extend into the nose cone 300, the distal end portions of these pull wires can be configured to be rotationally symmetric about the distal end portion 302d of the guide wire shaft. Each distal end portion of the pull wire can have a proximal segment embedded with the nose cone and a distal segment extending outside the nose cone.

As shown in FIGS. 17A-17B, the distal end portions 324, 326 of the pull wires are nonparallel to the distal end portion 302d of the guide wire shaft. By allowing the distal end portions 324, 326 of the pull wires to extend out of the nose cone through corresponding side openings 330, 332, the radial distance between the distal end portions 324, 326 of the pull wires and the distal end portion 302d of the guide wire shaft can be further increased (compared to the example of FIGS. 16A-16B) because such radial distance is not limited by the diameter of the nose cone. As a result, the pulling angle θ between the distal end portion 324 (or 326) of the pull wire and the distal end portion 302d of the guide wire shaft can be further increased, thereby allowing bending the nose cone 300A or 300B to a desired bend angle α with a smaller tension force applied to the pull wire 314 (or 316). Further, as described above, the required tension force to bend the distal portion 308 and/or the transition portion 310 (relative to the proximal portion 306) is further reduced compared to bending the entire nose cone and the guide wire shaft extending therethrough.

In the examples of FIGS. 16A-17E, a delivery apparatus including nose cone 300 (or nose cone 300A or 300B) with one or more pull wires 314, 316, can also include a pull wire 78 for adjusting the curvature of shaft 22, as described above. The curvature of the shaft 22 can be independently adjusted via pull wire 78 relative to the adjustment of the curvature of the nose cone 300 (or nose cone 300A or 300B). In some examples, a delivery apparatus including nose cone 300 (or nose cone 300A or 300B) with one or more pull wires 314, 316, can omit the pull wire 78 for adjusting the curvature of shaft 22, in which case the delivery apparatus can be steered through a patient's vasculature via adjustment of the curvature of the nose cone.

Moreover, nose cones 300, 300A, 300B can be modified to include any of the features of the nose cone 32, and vice versa. For example, any of nose cones 300, 300A, 300B can be modified to include a shoulder portion 126 that extends inside of balloon 28.

### Sterilization

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

### Additional Examples of the Disclosed Technology

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A delivery apparatus for implanting a prosthetic valve, the delivery apparatus comprising: a handle; a first shaft extending distally from the handle; a second shaft extending through a lumen of the first shaft; a nose cone mounted on a distal end portion of the second shaft; a balloon mounted along the distal end portion of the second shaft; and a first pull wire extending longitudinally along the second shaft, wherein a distal end of the first pull wire is fixed relative to the second shaft at a location that is distal to a distal end of the balloon, wherein tension in the first pull wire can be adjusted to adjust a curvature of the distal end portion of the second shaft and the nose cone.

Example 2. The delivery apparatus of any example herein, particularly example 1, wherein the nose cone is connected to a shoulder portion that extends into the balloon.

Example 3. The delivery apparatus of any example herein, particularly any one of examples 1-2, wherein the distal end of the first pull wire is fixedly connected to a ring located at a distal end of the nose cone.

Example 4. The delivery apparatus of any example herein, particularly any one of examples 1-3, wherein the second shaft has a guide wire lumen configured to receive a guide wire and a side lumen radially offset from the guide wire lumen, wherein the first pull wire extends through the side lumen at least partially along the length of the first pull wire.

Example 5. The delivery apparatus of any example herein, particularly any one of examples 1-4, wherein at least a portion of the second shaft comprises a braided layer.

Example 6. The delivery apparatus of any example herein, particularly example 5, wherein the braided layer has a braid density that is greater along the distal end portion of the second shaft than along a proximal end portion of the second shaft.

Example 7. The delivery apparatus of any example herein, particularly any one of examples 1-6, wherein a proximal end of the first pull wire is operatively coupled to an adjustment mechanism configured to adjust the tension in the first pull wire.

Example 8. The delivery apparatus of any example herein, particularly example 7, wherein the adjustment mechanism is incorporated into a Y-connector positioned proximal to the handle, wherein the Y-connector comprises a first actuation member configured to move the first pull wire axially relative to the handle, thereby adjusting the curvature of the distal end portion of the second shaft and the nose cone through a tensile force applied to the distal end of the first pull wire, wherein the Y-connector further comprises a locking mechanism configured to lock an axial position of the proximal end of the first pull wire.

Example 9. The delivery apparatus of any example herein, particularly example 8, wherein the first actuation mechanism comprises a sliding member that is axially movable within a channel of the Y-connector, wherein the proximal end of the first pull wire is connected to a post of the sliding member, wherein the locking mechanism comprises a plurality of recesses distributed along the channel of the Y-connector, wherein the plurality of recesses are configured to receive one or more protruding pins of the sliding member.

Example 10. The delivery apparatus of any example herein, particularly any one of examples 1-9, further comprising a third shaft extending distally from the handle and a second pull wire extending longitudinally along the third shaft, wherein the first shaft extends through a lumen of the third shaft, wherein a distal end of the second pull wire is connected to a distal end portion of the third shaft, wherein tension in the second pull wire can be adjusted to adjust a curvature of the distal end portion of the third shaft.

Example 11. The delivery apparatus of any example herein, particularly example 10, wherein the first shaft comprises a valve retaining region configured to mount a prosthetic valve thereto, wherein a proximal end of the valve retaining region is distal to the distal end of the second pull wire.

Example 12. The delivery apparatus of any example herein, particularly any one of examples 10-11, wherein the handle comprises a second actuation mechanism configured to adjust tension of the second pull wire, thereby adjusting the curvature of the distal end portion of the third shaft.

Example 13. The delivery apparatus of any example herein, particularly any one of examples 10-12, wherein the third shaft and the handle are configured to be movable in an axial direction relative to the first shaft and the second shaft.

Example 14. A delivery apparatus for implanting a prosthetic valve, the delivery apparatus comprising: a handle; a first shaft extending distally from the handle; a second shaft extending through a lumen of the first shaft; a third shaft extending through a lumen of the second shaft; a nose cone mounted on a distal end portion of the third shaft; a first pull wire extending longitudinally along the third shaft; and a second pull wire extending longitudinally along the first shaft, wherein a distal end of the first pull wire is fixedly connected to the nose cone and a distal end of the second pull wire is fixedly connected to a distal end portion of the first shaft, wherein tensioning the first pull wire is configured to adjust a curvature of the nose cone, wherein tensioning the second pull wire is configured to adjust a curvature of the distal end portion of the first shaft, wherein the distal end of the first pull wire is distal to the distal end of the second pull wire.

Example 15. The delivery apparatus of any example herein, particularly example 14, further comprising a balloon mounted along the distal end portion of the third shaft, wherein the distal end of the first pull wire is distal to a distal end of the balloon.

Example 16. The delivery apparatus of any example herein, particularly any one of examples 14-15, wherein the distal end of the first pull wire is fixedly connected to a pull ring located at a distal tip of the nose cone.

Example 17. The delivery apparatus of any example herein, particularly any one of examples 14-16, wherein the distal end portion of the third shaft comprises a braided layer that extends into a lumen of the nose cone.

Example 18. The delivery apparatus of any example herein, particularly any one of examples 14-17, further comprising a first actuator connected to a proximal end of the first pull wire and a second actuator connected to a proximal end of the second pull wire, wherein actuation of the first actuator is configured to adjust the curvature of the nose cone, wherein actuation of the second actuator is configured to adjust the curvature of the distal end portion of the first shaft.

Example 19. A method comprising: receiving a delivery apparatus comprising: a handle; a first shaft extending distally from the handle; a second shaft extending through a lumen of the first shaft; a third shaft extending through a lumen of the second shaft; a nose cone mounted on a distal end portion of the third shaft; a first pull wire extending longitudinally along the third shaft; and a second pull wire extending longitudinally along the first shaft; increasing tension in the second pull wire to adjust a curvature of the distal end portion of the first shaft; and increasing tension in the first pull wire to adjust a curvature of the nose cone.

Example 20. The method of any example herein, particularly example 19, further comprising inflating a balloon mounted along the distal end portion of the third shaft, wherein a distal end of the first pull wire is distal to a distal end of the balloon, wherein a distal end of the second pull wire is proximal to a proximal end of the balloon.

Example 21. A method of implanting a prosthetic valve, the method comprising: introducing a delivery apparatus into a vasculature of a subject, wherein the prosthetic valve is in a radially compressed configuration on a distal end portion of a first shaft of the delivery apparatus; adjusting a curvature of a distal end portion of a second that extends over the first shaft to navigate the prosthetic valve through an arched region of the vasculature; positioning the prosthetic valve within or adjacent an annulus of a native heart valve; retracting the second shaft relative to the prosthetic valve and a balloon of the delivery apparatus; adjusting a curvature of a nose cone of the delivery apparatus until the prosthetic valve is substantially coaxial with the annulus of the native heart valve; and inflating the balloon to radially expand the prosthetic valve.

Example 22. The method of any example herein, particularly example 21, wherein adjusting the curvature of the distal end portion of the second shaft comprises tensioning a first pull wire, wherein a distal end of the first pull wire is fixedly connected to the distal end portion of the second shaft, wherein adjusting the curvature of the nose cone comprises tensioning a second pull wire, wherein a distal end of the second pull wire is fixedly connected to the nose cone, wherein the distal end of the first pull wire is proximal to the prosthetic valve, wherein the distal end of the second pull wire is distal to the prosthetic valve.

Example 23. The method of any example herein, particularly any one of examples 21-22, wherein tensioning the first pull wire comprises actuating a first actuator connected to a proximal end of the first pull wire, wherein tensioning the second pull wire comprises actuating a second actuator connected to a proximal end of the second pull wire.

Example 24. A delivery apparatus for implanting a prosthetic valve, the delivery apparatus comprising: a handle; a shaft extending distally from the handle; a nose cone mounted on a distal end portion of the shaft; and a pull wire extending longitudinally along the shaft, wherein a distal end of the pull wire is fixedly connected to a distal end of the nose cone, wherein tension in the first pull wire can be adjusted to adjust a curvature of the distal end portion of the shaft and the nose cone.

Example 25. The delivery apparatus of any example herein, particularly example 24, wherein the shaft is a first shaft; the delivery apparatus further comprising a second shaft extending distally from the handle, wherein the first shaft extends through a lumen of the second shaft.

Example 26. The delivery apparatus of any example herein, particularly example 25, wherein the pull wire is a first pull wire and the delivery apparatus further comprises a third shaft extending distally from the handle and a second pull wire extending longitudinally along the third shaft, wherein the second shaft extends through a lumen of the third shaft, wherein a distal end of the second pull wire is connected to a distal end portion of the third shaft, wherein tension in the second pull wire can be adjusted to adjust a curvature of the distal end portion of the third shaft.

Example 27. The delivery apparatus of any example herein, particularly example 26, wherein the second shaft comprises a valve retaining region configured to mount a prosthetic valve thereto, wherein a proximal end of the valve retaining region is distal to the distal end of the second pull wire.

Example 28. The delivery apparatus of any example herein, particularly example 24, further comprising a balloon mounted on the distal end portion of the shaft, wherein the distal end of the pull wire is positioned distal to a distal end of the balloon.

Example 29. The delivery apparatus of any example herein, particularly example 28, wherein the nose cone is connected to a shoulder portion extending into the balloon.

Example 30. The delivery apparatus of any example herein, particularly example 29, wherein the shaft comprises a braided layer which extends through the shoulder portion and into a lumen of the nose cone, wherein the braided layer in the nose cone has a higher braid density than the braided layer in the shoulder portion.

Example 31. A delivery apparatus for implanting a prosthetic implant, the delivery apparatus comprising: a handle; a shaft extending distally from the handle; a nose cone mounted on a distal end portion of the shaft; and a pull wire extending longitudinally along the shaft and into the nose cone, wherein a distal end portion of the pull wire is nonparallel to the distal end portion of the shaft.

Example 32. The delivery apparatus of any example herein, particularly example 31, wherein the nose cone comprises a distal portion and a proximal portion, wherein tensioning the pull wire is configured to bend the distal portion of the nose cone relative to the proximal portion of the nose cone while keeping the proximal portion of the nose cone substantially straight.

Example 33. The delivery apparatus of any example herein, particularly example 32, wherein tensioning the pull wire is configured to bend the distal portion of the nose cone up to 90 degrees relative to the proximal portion of the nose cone.

Example 34. The delivery apparatus of any example herein, particularly any one of examples 32-33, wherein the proximal portion of the nose cone has an axial length that is between 5 mm and 8 mm, inclusive.

Example 35. The delivery apparatus of any example herein, particularly any one of examples 32-34, wherein the distal portion of the nose cone has a lower flexural modulus than the proximal portion of the nose cone.

Example 36. The delivery apparatus of any example herein, particularly example 35, wherein the distal portion of the nose cone comprises a first material and the proximal portion of the nose cone comprises a second material that is different from the first material.

Example 37. The delivery apparatus of any example herein, particularly any one of examples 32-34, wherein the distal portion and the proximal portion of the nose cone are connected by a flexible portion of the nose cone, wherein the flexible portion of the nose cone has a lower flexural modulus than the proximal portion of the nose cone.

Example 38. The delivery apparatus of any example herein, particularly example 37, wherein the flexible portion of the nose cone comprises a first material and the proximal portion of the nose cone comprises a second material that is different from the first material.

Example 39. The delivery apparatus of any example herein, particularly any one of examples 31-38, wherein the distal end portion of the pull wire extends through a channel within the nose cone, wherein the channel extends alongside an axial profile of the nose cone.

Example 40. The delivery apparatus of any example herein, particularly any one of examples 32-38, wherein the proximal portion of the nose cone comprises a side opening, wherein the distal end portion of the pull wire extends out of the nose cone through the side opening and connects to the distal portion of the nose cone.

Example 41. The delivery apparatus of any example herein, particularly any one of examples 31-40, wherein the pull wire is one of a plurality of pull wires, each pull wire having a corresponding distal end portion extending into the nose cone, wherein the distal end portions of the plurality of pull wires are rotationally symmetric about the distal end portion of the shaft.

Example 42. The delivery apparatus of any example herein, particularly example 41, wherein the pull wire is a first pull wire, wherein the delivery apparatus further comprises a second pull wire, wherein the distal end portions of the first pull wire and the second pull wire are in mirrored positions relative to the distal end portion of the shaft.

Example 43. The delivery apparatus of any example herein, particularly any one of examples 31-33, wherein a distal end of the pull wire is fixedly connected to a ring located at a distal end of the nose cone.

Example 44. A delivery apparatus for implanting a prosthetic implant, the delivery apparatus comprising: a handle; a shaft extending distally from the handle; a nose cone mounted on a distal end portion of the shaft; and a pull wire extending longitudinally along the shaft and into the nose cone, wherein the nose cone comprises a distal portion and a proximal portion, wherein a distal end portion of the pull wire extends out of the proximal portion of the nose cone and connects to the distal portion of the nose cone.

Example 45. The delivery apparatus of any example herein, particularly example 44, wherein tensioning the pull wire is configured to bend the distal portion of the nose cone relative to the proximal portion of the nose cone while keeping the proximal portion of the nose cone substantially straight.

Example 46. The delivery apparatus of any example herein, particularly example 45, wherein tensioning the pull wire is configured to bend the distal portion of the nose cone up to 90 degrees relative to the proximal portion of the nose cone.

Example 47. The delivery apparatus of any example herein, particularly any one of examples 44-46, wherein the pull wire is one of a plurality of pull wires, each pull wire having a corresponding distal end portion extending into the nose cone, wherein the distal end portions of the plurality of pull wires are rotationally symmetric about the distal end portion of the shaft.

Example 48. A delivery apparatus for implanting a prosthetic implant, the delivery apparatus comprising: a handle; a shaft extending distally from the handle; a nose cone mounted on a distal end portion of the shaft; and a pull wire extending longitudinally along the shaft and into the nose cone, wherein the nose cone comprises a distal portion and a proximal portion, wherein a distal end portion of the pull wire extends through a channel within the nose cone, wherein a radial distance of the channel from a central longitudinal axis of the nose cone varies along the length of the channel

Example 49. The delivery apparatus of any example herein, particularly example 48, wherein tensioning the pull wire is configured to bend the distal portion of the nose cone relative to the proximal portion of the nose cone while keeping the proximal portion of the nose cone substantially straight.

Example 50. The delivery apparatus of any example herein, particularly example 49, wherein tensioning the pull wire is configured to bend the distal portion of the nose cone up to 90 degrees relative to the proximal portion of the nose cone.

Example 51. The delivery apparatus of any example herein, particularly any one of examples 48-50, wherein the channel extends along a curved path through the nose cone that corresponds to an axial profile of the nose cone.

Example 52. A delivery apparatus for implanting a prosthetic device, the delivery apparatus comprising: a handle; a shaft extending distally from the handle; a nose cone mounted on a distal end portion of the shaft; and a pull wire extending longitudinally along the shaft and into the nose cone, wherein the nose cone comprises a distal portion and a proximal portion, wherein tensioning the pull wire is configured to bend the distal portion of the nose cone relative to the proximal portion of the nose cone up to 90 degrees while keeping the proximal portion of the nose cone substantially straight, wherein the proximal portion of the nose cone has an axial length between 5 mm and 8 mm, inclusive.

Example 53. The delivery apparatus of any example herein, particularly example 52, wherein a distal end portion of the pull wire extends through a channel within the nose cone, wherein the channel extends alongside an axial profile of the nose cone.

Example 54. The delivery apparatus of any example herein, particularly example 52, wherein a distal end portion of the pull wire extends out of the proximal portion of the nose cone and connects to the distal portion of the nose cone.

Example 55. The delivery apparatus of any example herein, particularly any one of examples 52-54, wherein the pull wire is one of a plurality of pull wires, each pull wire having a corresponding distal end portion extending into the nose cone, wherein the distal end portions of the plurality of pull wires are rotationally symmetric about the distal end portion of the shaft.

Example 56. A method of implanting a prosthetic device, the method comprising: introducing a delivery apparatus into a vasculature of a subject, wherein the prosthetic device is mounted on the delivery apparatus, wherein the delivery apparatus comprises a handle, a shaft extending distally from the handle, a nose cone mounted on a distal end portion of the shaft, and a pull wire extending longitudinally along the shaft and into the nose cone; and tensioning the pull wire to bend the nose cone, wherein the tensioning bends a distal portion of the nose cone relative to a proximal portion of the nose cone while keeping the proximal portion of the nose cone substantially straight.

Example 57. The method of any example herein, particularly any one of examples 20-23 and 56, wherein the subject is a human patient or a non-living simulation object.

Example 58. A method comprising sterilizing the delivery apparatus of any example herein, particularly any one of examples 1-17 and 24-55.

The features described herein with regard to any example can be combined with other features described in any one or more of the other examples, unless otherwise stated. For example, any one or more of the features of one delivery apparatus can be combined with any one or more features of another delivery apparatus.

It is to be understood that the treatment techniques, methods, steps, etc. described or suggested herein or in references incorporated herein can be performed on a living animal or on a non-living simulation, such as on a cadaver, cadaver heart, anthropomorphic ghost, simulator (e.g., with the body parts, tissue, etc. being simulated), etc.

In view of the many possible examples to which the principles of the disclosed technology may be applied, it should be recognized that the illustrated examples are only preferred examples of the technology and should not be taken as limiting the scope of the disclosure. Rather, the scope of the claimed subject matter is defined by the following claims and their equivalents.

What is claimed is:

1. A delivery apparatus for implanting a prosthetic valve, the delivery apparatus comprising:
a handle;
a first shaft extending distally from the handle;
a second shaft extending through a lumen of the first shaft;
a nose cone mounted on a distal end portion of the second shaft;
a balloon mounted along the distal end portion of the second shaft; and
a first pull wire extending longitudinally along the second shaft,
wherein a distal end of the first pull wire is fixed relative to the second shaft at a location that is distal to a distal end of the balloon, wherein tension in the first pull wire can be adjusted to adjust a curvature of the distal end portion of the second shaft and the nose cone.

2. The delivery apparatus of claim 1, wherein the nose cone is connected to a shoulder portion that extends into the balloon.

3. The delivery apparatus of claim 1, wherein the distal end of the first pull wire is fixedly connected to a ring located at a distal end of the nose cone.

4. The delivery apparatus of claim 1, wherein the second shaft has a guide wire lumen configured to receive a guide wire and a side lumen radially offset from the guide wire lumen, wherein the first pull wire extends through the side lumen at least partially along the length of the first pull wire.

5. The delivery apparatus of claim 1, wherein at least a portion of the second shaft comprises a braided layer.

6. The delivery apparatus of claim 5, wherein the braided layer has a braid density that is greater along the distal end portion of the second shaft than along a proximal end portion of the second shaft.

7. The delivery apparatus of claim 1, wherein a proximal end of the first pull wire is operatively coupled to an adjustment mechanism configured to adjust the tension in the first pull wire.

8. The delivery apparatus of claim 7, wherein the adjustment mechanism is incorporated into a Y-connector positioned proximal to the handle, wherein the Y-connector comprises a first actuation member configured to move the first pull wire axially relative to the handle, thereby adjusting the curvature of the distal end portion of the second shaft and the nose cone through a tensile force applied to the distal end of the first pull wire, wherein the Y-connector further comprises a locking mechanism configured to lock an axial position of the proximal end of the first pull wire.

9. The delivery apparatus of claim 8, wherein the first actuation mechanism comprises a sliding member that is axially movable within a channel of the Y-connector, wherein the proximal end of the first pull wire is connected to a post of the sliding member, wherein the locking mechanism comprises a plurality of recesses distributed along the channel of the Y-connector, wherein the plurality of recesses are configured to receive one or more protruding pins of the sliding member.

10. The delivery apparatus of claim 1, further comprising a third shaft extending distally from the handle and a second pull wire extending longitudinally along the third shaft,
wherein the first shaft extends through a lumen of the third shaft,
wherein a distal end of the second pull wire is connected to a distal end portion of the third shaft, wherein tension in the second pull wire can be adjusted to adjust a curvature of the distal end portion of the third shaft.

11. The delivery apparatus of claim 10, wherein the first shaft comprises a valve retaining region configured to mount a prosthetic valve thereto, wherein a proximal end of the valve retaining region is distal to the distal end of the second pull wire.

12. The delivery apparatus of claim 10, wherein the handle comprises a second actuation mechanism configured to adjust tension of the second pull wire, thereby adjusting the curvature of the distal end portion of the third shaft.

13. The delivery apparatus of claim 10, wherein the third shaft and the handle are configured to be movable in an axial direction relative to the first shaft and the second shaft.

14. A delivery apparatus for implanting a prosthetic valve, the delivery apparatus comprising:
a handle;
a shaft extending distally from the handle;
a nose cone mounted on a distal end portion of the shaft; and
a pull wire extending longitudinally along the shaft,
wherein a distal end of the pull wire is fixedly connected to a distal end of the nose cone, wherein tension in the first pull wire can be adjusted to adjust a curvature of the distal end portion of the shaft and the nose cone.

15. The delivery apparatus of claim 14, wherein the shaft is a first shaft; the delivery apparatus further comprising a second shaft extending distally from the handle, wherein the first shaft extends through a lumen of the second shaft.

16. The delivery apparatus of claim 15, wherein the pull wire is a first pull wire and the delivery apparatus further comprises a third shaft extending distally from the handle and a second pull wire extending longitudinally along the third shaft, wherein the second shaft extends through a lumen of the third shaft, wherein a distal end of the second pull wire is connected to a distal end portion of the third shaft, wherein tension in the second pull wire can be adjusted to adjust a curvature of the distal end portion of the third shaft.

17. The delivery apparatus of claim 14, further comprising a balloon mounted on the distal end portion of the shaft, wherein the distal end of the pull wire is positioned distal to a distal end of the balloon.

18. A delivery apparatus for implanting a prosthetic implant, the delivery apparatus comprising:
a handle;
a shaft extending distally from the handle;
a nose cone mounted on a distal end portion of the shaft; and
a pull wire extending longitudinally along the shaft and into the nose cone,
wherein a distal end portion of the pull wire is nonparallel to the distal end portion of the shaft.

19. The delivery apparatus of claim 18, wherein the nose cone comprises a distal portion and a proximal portion, wherein tensioning the pull wire is configured to bend the distal portion of the nose cone relative to the proximal portion of the nose cone while keeping the proximal portion of the nose cone substantially straight.

20. The delivery apparatus of claim 19, wherein tensioning the pull wire is configured to bend the distal portion of the nose cone up to 90 degrees relative to the proximal portion of the nose cone.
